(19) United States
(12) Patent Application Publication
(10) Pub. No.: US 2014/0234373 A1
(43) Pub. Date: Aug. 21, 2014

(54) Methods of Promoting Immune Tolerance

(71) Applicant: Georgia Regents University, August, GA (US)

(72) Inventors: Andrew L. Mellor, Augusta, GA (US); David H. Munn, Augusta, GA (US); Lei Huang, Evans, GA (US); Madhav Sharma, Augusta, GA (US)

(73) Assignee: Georfia Regents University, August, GA (US)

(21) Appl. No.: 14/344,029

(22) PCT Filed: Sep. 17, 2012
(86) PCT No.: PCT/US2012/055750

§ 371 Date: Mar. 10, 2014

Related U.S. Application Data
(60) Provisional application No. 61/585,076, filed on Jan. 10, 2012.
(60) Provisional application No. 61/535,736, filed on Sep. 16, 2011.

(51) Int. Cl. A61K 47/34; A61K 39/02

(52) U.S. Cl. CPC A61K 47/34; A61K 39/02; A61K 2039/52
USPC 424/234.1; 424/184.1

(57) ABSTRACT

Compositions including a polynucleotide combined with a vehicle and methods of their use to induce a suppressive immune response are provided. In some embodiments the compositions induce an increase in expression of indoleamine 2,3 dioxygenase (IDO) enzyme activity in cells. The methods and compositions can be used to inhibit or reduce immune-mediated tissue destruction, to treat autoimmune diseases and inflammatory responses, to promote immune tolerance, to enhance tolerizing vaccines, to treat allergies, to treat asthma, or to enhance mucosal tolerance in subject. Methods and compositions for inducing a suppressive immune response for while minimizing undesirable side effects in the subject are also provided. An exemplary undesirable side effect is systemic release of INFγ. Exemplary compositions that can be used to induce an immune response in a subject without inducing systemic release of INFγ include compositions containing a polynucleotide lacking an immunostimulatory nucleic acid sequence complexed with a carrier.

### STATEMENT REGARDING FEDERALLY SPONSORED RESEARCH OR DEVELOPMENT

This invention was made with Government Support under Agreement AI-075165 awarded to Andrew Mellor by the National Institute of Allergy and Infectious Diseases, National Institutes of Health. The Government has certain rights in the invention.

### FIELD OF THE INVENTION

The application generally relates to methods and compositions for modulating immune responses, in particular, methods and compositions for promoting, inducing or stimulating a suppressive immune response to treat syndromes in which the immune system damages healthy tissues due to loss of tolerance that allows excessive immunity.

### BACKGROUND OF THE INVENTION

Most autoimmune diseases do not have cures. Instead, doctors treat one or more symptoms of the autoimmune disease. For example, doctors prescribe corticosteroid drugs, non-steroidal anti-inflammatory drugs (NSAIDs) or more powerful immunosuppressant drugs such as cyclophosphamide, methotrexate and azathioprine to suppress the immune response and stop the progression of the disease. Radiation of the lymph nodes and plasmapheresis (a procedure that removes the diseased cells and harmful molecules from the blood circulation) are other ways of treating autoimmune diseases. These treatments are often insufficient and can include potentially toxic side effects.

Gene therapy is a relatively new method for treating autoimmune diseases and inflammatory responses. Gene therapy typically involves the insertion, alteration, or removal of genes within an individual's cells and biological tissues to treat disease. Nucleic acids containing new genes that encode therapeutic proteins—or that block specific gene expression in target cells—are delivered to a patient's cells using carriers such as cationic polymers. Cationic polymers form stable complexes (often referred to as ‘nanoparticles’ due to their typical size) with nucleic acids and can enhance the delivery efficiency of the nucleic acids to cells.

The size of the nucleic acid complexes can also be optimized to enhance delivery. For example, nanoparticles are known to effectively deliver nucleic acids to cells because cells readily ingest nanoparticles and then release the nucleic acids inside cells. Although cationic polymers offer a promising mechanism for delivering nucleic acids to cells, nanoparticles also stimulate rapid, systemic expression of pro-inflammatory cytokines such as interferon type II (IFNγ), an undesirable and potentially toxic side effect (Intra, J., and Salem A. K., J Control Release, 130:129-138 (2008)) in clinical settings where inhibiting hyper-immune responses that target healthy tissues in the therapeutic goal.

Under certain conditions, proinflammatory responses can promote effective immunity in hypo-immune syndromes such as cancer and chronic infections. For example, DNA nanoparticles formed with the cationic polymer polyethylenimine (PEI) stimulated rapid release of endogenous IL-12 by macrophages and enhanced Th1 effector T cell responses and anti-tumor immunity (Chen, et al., Biomaterials, 31:8172-8180 (2010)). Incorporation of nucleic acids encoding IL-12 into PEI nanoparticles was more effective than transiently induced endogenous IL-12 release in preventing lung metastases in a mouse model of lung cancer, though induction of endogenous IL-12 was also effective in slowing tumor growth in the absence of exogenous IL-12 encoded by bacterial plasmid DNA (pDNA) (Rodrigo-Garzon, et al., Cancer Gene Ther, 17:20-27 (2010)). These studies suggest that sustained expression of exogenous pro-inflammatory cytokine genes, and transient release of endogenous cytokines in response to nanoparticles loaded with expression vectors may be beneficial in treating clinical hypo-immune syndromes such as cancer and some chronic infectious diseases where host regulatory responses drive disease progression by inhibiting immune-mediated elimination of tumor cells and pathogen-infected cells, respectively.

However, systemic release of proinflammatory cytokines induced by nanoparticles loaded with expression vectors have undesirable toxicities that preclude wide clinical application of sustained nanoparticle-based gene therapy, particularly as it pertains to treatment of autoimmune diseases and inflammatory disorders. For example, nanoparticles loaded with expression vectors can induce activation of immune helper/effector cells leading to pathological effects on healthy tissues, and a reduction in the regulatory barriers that prevent autoimmunity.

Therefore, it is an object of the invention to provide methods and compositions for inhibiting or reducing immune cell responses.

It is another object of the invention to provide methods and compositions for inducing or promoting immune tolerance.

It is a further object of the invention to provide methods and compositions for inhibiting or reducing immune cell responses with reduced systemic side effects relative to a control.

It is another object of the invention to provide methods and compositions for treating one or more symptoms of an immune disorder with compositions that have reduced systemic side effects.

### SUMMARY OF THE INVENTION

Methods and compositions to combine polynucleotides with vehicles for inducing a regulatory immune response are provided. An exemplary regulatory response is a suppressive response. In certain embodiments, the disclosed compositions can activate or induce immune cells to promote a suppressive immune response. In preferred embodiments, the compositions induce or promote an increase in expression of indoleamine 2,3 dioxygenase (IDO) enzyme activity in cells. In still other preferred embodiments, differentiation, activation, or proliferation of effector T cells is reduced relative to a control. The compositions can recruit or induce immune cells with regulatory phenotypes including, but not limited to antigen presenting cells, T cells, natural killer cells, mesynchemal stem cells (MSCs), and myeloid-derived suppressor cells (MDSCs). Suppressor functions of Tregs include increased proliferation or local accumulation of Tregs at lesion sites, reduced proliferation of helper/effector T cell precursors and consequent reduced differentiation into functional helper/effector T cells, and enhanced production of IL-10, IL-2 and TGF-β.

The methods and compositions can be used to inhibit immune-mediated tissue destruction, to treat autoimmune diseases and inflammatory responses, including but not limited to rheumatoid arthritis and type I diabetes, lupus (SLE), to enhance tolerizing vaccines, to treat allergies, to treat asthma, or to enhance mucosal tolerance.

In certain embodiments, the methods and compositions for inducing a regulatory immune response have reduced or limited undesirable side effects compared to existing therapies. Exemplary undesirable side effects include, but are not limited to a systemic inflammatory response. It has been discovered that the signaling transduction mechanisms involved in promoting regulatory immune responses in a subject can be decoupled from signal transduction mechanisms involved in promoting inflammatory responses. Therefore, methods and compositions are provided that induce a regulatory response, preferably a suppressive immune response, in a subject without promoting a systemic pro-inflammatory (immune stimulatory) response or having a substantially reduced systemic inflammatory response relative to controls. Exemplary methods for inducing a regulatory immune response in a subject include administering to the subject an effective amount of a composition that induces a suppressive immune response in the subject without inducing systemic release of one or more proinflammatory cytokines in the subject. In one embodiment, systemic release of INFγ is reduced compared to a control.

Exemplary compositions that can be used to induce a regulatory immune response in a subject without inducing systemic release of one or more proinflammatory cytokines includes, but is not limited to compositions containing one or more polynucleotides combined with a vehicle, for example a carrier, to form nanoparticle compositions. The polynucleotide can be a tolerogenic polynucleotide that when complexed with a carrier, and administered to a subject induces an immune suppressive response, such as immune tolerance.

One embodiment provides a composition containing a linear form of the cationic polyamine polyethylenimine (PEI) suspended in sterile, pyrogen-free water at a concentration of 150 millimolar nitrogen residues diluted with 200 microliter of 5% glucose at room temperature and added to 21 to 30 micrograms of double-stranded bacterial plasmid DNA of between 2 to 20 kilobasepairs in length pre-diluted in 5% glucose to give a final nitrogen residues:nucleic acid phosphate (N:P) ratio of 10 to 18.

In some embodiments the polynucleotide increases expression of IDO in IDO-competent cells in vivo or ex vivo. In still other embodiments, the polynucleotides in which immunostimulatory elements are reduced, absent, or masked can induce expression of IDO without stimulating systemic release (defined as post-treatment increase in serum levels above basal levels in patients or animals before treatment) of pro-inflammatory cytokines such as IFNγ. The polynucleotide typically does not contain ligands for toll-like receptors (TLRs) or related receptors or contains ligands for TLRs that have been treated so that they do not activate the toll-like receptor signal transduction pathway or are otherwise functionally inert. For example masked the TLRs can be masked or otherwise covered. Exemplary receptors that bind nucleic acids include, but are not limited to toll-like receptors (TLR)3, TLR7, and TLR9. Exemplary toll-like receptor ligands include un-methylated CpG motifs that bind to TLR9 to activate innate immune cells. In some embodiments, the polynucleotides do not contain ligands for specified TLRs that trigger immune cell activation and consequent release of pro-inflammatory cytokines, but are able to bind to other receptors that sense the presence of nucleic acids inside cells that ingest DNA nanoparticles. If the polynucleotides do contain ligands for toll-like receptors, the ligands for the toll-like receptors are masked or modified to render them non-functional.

The polynucleotide can be combined with a vehicle for example a carrier suitable for delivering polynucleotides. The vehicle can optimize delivery, uptake or both of the polynucleotide to cells, preferably immune cells. The vehicle can be a polymer or co-polymer. Preferred vehicles include, but are not limited to particulate carriers. The particles can be microparticles, nanoparticles, or a combination thereof. Preferred particles are composed of the cationic polymer polyethyenimine (PEI) and polynucleotides to form nanoparticles.

Another embodiment provides a method for inducing a regulatory immune response in a subject by stimulating expression of indoleamine 2,3 dioxygenase (IDO) enzyme activity inside cells to induce a suppressive immune response in the subject wherein the expression of IDO enzyme activity is dependent on interferons (IFNs) produced in response to the treatment, and independent of signaling via TLR9. The methods include administering an effective amount of a polynucleotide combined with a vehicle to stimulate IFN-dependent and TLR9-independent expression of IDO enzyme in cells. IDO expression can result in multiple effects, including the inhibition of T-cell proliferation, increased T-cell apoptosis, and de novo induction and activation of Tregs, which all lead to an impairment of the cellular immune response, i.e., a suppressive immune response that promotes immune tolerance. Cells that can be stimulated to express IDO include but are not limited to antigen presenting cells including dendritic cells and macrophages, fibroblasts and epithelial cells. Dendritic cells that express IDO include cells that display attributes of plasmacytoid DCs (pDCs) in mice and humans; in mice IDO-competent DCs display attributes of both pDCs and B cells, and express B220, CD19, Pax5, CD11c CD8α, or combinations thereof. Though local IFN type 1 (IFNα) and type II (IFNγ) may be necessary to stimulate DCS to express IDO, systemic IFNγ release induced by the preferred embodiments of DNA/PEI nanoparticles lacking TLR ligands is typically reduced substantially, or is negligible compared to a control containing bacterial plasmid DNA (pDNA) complexed with PEI, which contain TLR9 ligands. In this embodiment, an exemplary control includes nanoparticles combined with immunostimulatory nucleic acids that elicit systemic IFNγ release.

In preferred embodiments, expression of IDO may be IFN type I independent, but in other embodiments IDO induction may be IFN type I dependent.

### BRIEF DESCRIPTION OF THE DRAWINGS

FIG. 1 is a hypothetical model of early responses to DNA/PEI nanoparticles (DNPs) that stimulate rapid NK cell activation, and IDO up-regulation in other innate immune cells. The model also depicts how these early responses to DNPs may cause dendritic cells (DCs) and regulatory T cells (Tregs) to acquire potent regulatory phenotypes that create local immune privilege, which inhibits hyper-immunity that drives autoimmune disease progression.

FIG. 2 shows a Western blot (FIG. 2A) and bar graphs (FIGS. 2B-D) indicating that IDO is induced rapidly in mucosal (lung, colon) and lymphoid (spleen, lymph nodes, LNs) tissues of B6 mice following treatment (24 hrs.) with pDNA/PEI nanoparticles (30 ng pDNA, N:P ratio 1:10, i/v). FIGS. 2B, 2C, and 2D show IDO activity (detected as the presence of the tryptophan catabolite kynurenine, kyn) in lymph nodes (B), lungs (C), and colon (D) tissues, of B6 (wild-type) and control IDO1 knockout (IDO1-KO) mice following treatment (24 hrs.) with pDNA/PEI nanoparticles (30 ng pDNA, N:P ratio 1:10, i/v).

FIG. 3A is a diagram depicting an experimental design to test the effect of pDNA/PEI nanoparticle treatment on antigen-specific T cell responses elicited in vivo in inguinal lymph nodes (dLNs) draining sites of immunization with cells expressing chicken ovalbumin (OVA). FIG. 3B are histograms showing FACS analysis of gated OVA-specific donor OT-2 T cells (labeled initially with the tracking dye CFSE, x-axis) 120 hours after OVA treatment from B6 mice treated as follows; no OVA immunization (left panel), OVA immunization +no nanoparticle (“no therapy”, second panel from left), OVA immunization +pDNA/PEI (third panel from left, and OVA immunization +pDNA/PEI+1MT (right panel) groups. FIG. 3C are dot plots showing cells positive for IFNγ (y-axis) gated dLN OT-2 T cells for no OVA, OVA+no nanoparticle (“no therapy”), OVA+pDNA/PEI, and OVA+pDNA/PEI+1MT groups 120 hours after OVA treatment (as detailed above). Absolute numbers of divided/undivided (CFSElow/high) OT-2 T cells are indicated below histograms. Proportions of OT-2 T cells expressing IFNγ are indicated on dot plots. Data are representative of three experiments. These data show that pDNA/PEI treatment resulted in >99% suppression (1,250/153,450×100%) of local OT-2 T cell proliferative responses to OVA immunization in dLNs, and that the suppressive effects of DNP treatment were abrogated in mice given IDO inhibitor (1MT).

The diagram at the top of FIG. 4 depicts assay procedures designed to test the effect of pDNA/PEI nanoparticle treatment on immune regulatory phenotypes of splenic dendritic cells (DCs) and Tregs. FIG. 4A is a line graph showing T cell proliferation (y-axis, thymidine incorporation as CPM×10⁻³) in cultures containing graded numbers of CD11c+DCs (x-axis, 6/12/25/50×10³ DCs) isolated from pDNA/PEI treated B6 (wild-type) mice, mixed with responder OVA-specific (OT-1) T cells, with (closed symbols) or without (open symbols) 1MT. Similarly, FIGS. 4B, 4C, 4D are line graphs showing OT-2 T cell proliferative responses elicited ex vivo by splenic DCs from pDNA/PEI treated mice with defective IDO 1 (IDO1-KO), interferon type II (IFNγR-KO) and type I (IFNAR-KO) genes, respectively. These data show that—as in pDNA/PEI treated B6 mice—DCs from pDNA/PEI treated IFNγR-KO mice suppressed OT-2 responses unless IDO inhibitor was present during culture; these data indicated that DCs from B6 and IFNγR-KO mice acquired potent regulatory phenotypes following pDNA/PEI treatment. However, DCs from pDNA/PEI treated IDO1-KO and IFNAR-KO mice stimulated robust OT-2 proliferation even when 1MT was not present, and adding 1MT did not further enhance OT-2 responses; these data indicated that DCs did not acquire potent regulatory phenotypes in IDO1-KO and IFNAR-KO following pDNA/PEI treatment. FIG. 4E is a line graph showing T cell proliferation (y-axis, thymidine incorporation as CPM×10⁻³) in cultures with graded numbers (x-axis, 2.5/5/10/20×10³ Tregs) splenic CD4+CD25+ Tregs isolated from pDNA/PEI treated B6 mice, mixed with responder H-Y-male-antigen specific A1 T cells & female antigen presenting cells (APCs) & cognate (male H-Y) peptides with (closed symbols) or without (open symbols) a cocktail of three mAbs to block PD-1 interactions with PD-L1 and PD-L2. Similarly, FIGS. 4F, 4G, 4H are line graphs showing A-1 T cell proliferative responses elicited ex vivo in the presence of splenic Tregs from pDNA/PEI treated mice with defective IDO1 (IDO1-KO), interferon type II (IFNγR-KO) and type I (IFNAR-KO) genes, respectively. These data show that—as in pDNA/PEI treated B6 mice—Tregs from pDNA/PEI treated IFNγR-KO mice suppressed A-1 T cell responses unless PD-1/PD-L blocking mAbs were present during culture. These data indicated that Tregs from B6 and IFNγR-KO mice acquired potent regulatory phenotypes following pDNA/PEI treatment, and that Treg activation to acquire regulatory phenotypes was mediated by IDO since PD-1/PD-L dependent suppression by Tregs is a hallmark feature of IDO-activated Tregs. However, Tregs from pDNA/PEI treated IDO1-KO and IFNAR-KO mice were not possess potent suppressor activity even when 20,000 Tregs were added to cultures, and adding PD-1/PD-L blocking mAbs did not enhance A1 T cell proliferation; these data indicated that Tregs did not acquire potent regulatory phenotypes in IDO1-KO and IFNAR-KO mice following pDNA/PEI treatment. Data shown are representative of experiments performed 2-3 times with graded numbers of DCs or Tregs.

FIG. 5A is a bar graph showing serum levels of IFNγ (ng/ml) in B6, TLR9-KO, and MyD88-KO mice with or without pDNA/PEI treatment. FIG. 5B is a bar graph showing serum levels of IFNγ (ng/ml) in B6 mice treated with pDNA/PEI, pDNA containing no un-methylated CpG motifs (TLR9 ligands) complexed with PEI (CpG-free/PEI), synthetic double-strand polydeoxyadenosine/thymidine polymers complexed with PEI (polyAT/PEI). These data show that systemic release of IFNγ after DNA nanoparticle treatment is dependent on the TLR9 signaling pathway in innate immune cells (FIG. 5A), and that removing TLR9 ligands from DNA nanoparticles eliminated these potentially toxic pro-inflammatory responses (FIG. 5B). FIG. 5C is a dot plot of splenocytes showing how NK cells were gated (selected) based on expression of the NK cell specific markers NK1.1 (y-axis), and DX5 (x-axis). FIG. 5D are histograms showing intracellular IFNγ detected in gated NK cells (NK1/1+DX5+) from untreated mice and mice treated for 3 hrs with pDNA/PEI and CpG^{free}pDNA/PEI nanoparticles and analyzed directly (ex vivo) or after further culture for 3 hr. with GolgiStop (+BFA) to permit accumulation of intracellular IFNγ. FIG. 5E is a bar graph showing serum IFN activity (U/ml) in an VSV-infection interference bio-assay with or without IFNγ neutralizing antibody. Data are representative of two or more experiments. These data show that NK cells were rapidly activated and uniformly expressed IFNγ in mice treated with DNA nanoparticles containing TLR9 ligands, while NK cells were not activated and did not express IFNγ in mice treated with DNA nanoparticles lacking TLR9 ligands.

FIG. 6 is a bar graph showing serum IFN activity (U/ml) with or without IFNγ neutralizing antibody in an VSV-infection interference bio-assay of DC2.4 cells that were untreated, or treated with pDNA/PEI, CpG-free/PEI, or pAT/PEI for 18 hrs. Data are representative of two or more experiments. These data show that nanoparticles stimulated DC2.4 cells to make IFN type I irrespective of whether DNA containing TLR9 ligands (un-methylated CpG motifs), though pAT/PEI nanoparticles stimulated substantially higher levels of IFN type I than the other nanoparticles used. FIGS. 7A-D show the effects of treating mice with the preferred embodiment (pAT/PEI nanoparticles) on experimentally induced joint arthritis using a mouse model of immune-mediated rheumatoid arthritis Lemos H. P. et al., Proc. Natl. Acad. Sci. (USA), 106:5954-5959 2009. FIG. 7A is a line graph indicating changes in knee thickness (mm) in this model 7 days after mBSA challenge to induce arthritis onset and treatment with vehicle (♦), pAT/PEI (◯), or pAT/PEI+1MT (). These data show that pAT/PEI treatment reduced knee swelling significantly, and that oral 1MT dosing abrogated this therapeutic effect. FIG. 7B is a bar graph indicating neutrophils (10⁴/cavity) infiltrating into joints 1 day after mBSA challenge. FIGS. 7C and 7D are bar graphs showing IL-6 (pg/ml) (C) and IL-17 (pg/ml) (D) cytokine levels in inflamed dLN cells assessed by multiplex analysis. Statistical significance was estimated by Student's t test. Data are representative of two experiments. These data show that pAT/PEI treatment reduced neutrophil infiltration and lowered levels of IL-6 and IL-17 expression in dLNs; for each parameter oral dosing with 1MT blocked he therapeutic effects of pAT/PEI treatment in this model of arthritis.

FIGS. 8A-E show tissue pathology, specifically safranin-O staining to detect the induced loss of proteoglycans present in healthy joints due to onset of autoimmune arthritis. FIG. 8A shows a healthy joint from an untreated B6 mouse; FIG. 8B shows a joint from a mouse treated to induce joint arthritis; FIG. 8C shows a joint from mouse treated to induced arthritis and given pAT/PEI treatment during the challenge phase to induce joint disease onset; FIG. 8D shows joint from a mouse treated as in 8C, but also given oral 1MT during the entire experiment; FIG. 8E shows a joint from an IDO1-deficient mouse treated as in 8C. These data show that joint injury—indicated by loss of proteoglycans—was prevented by pAT/PEI treatment and this therapeutic effect of pAT/PEI treatment was dependent on functional IDO.

FIG. 9 show line graphs indicating the incidence of type I diabetes (y-axis, %) over time α-axis, weeks) in type I diabetes prone non-obese female (NODf) mice treated with vehicle ((glucose 5%, n=10) (--)), pDNA/PEI (n=6 (-▪-)) from age 4-8 weeks (2-3 doses/week), or D1-MT (n=10 (-▴-). These data show that a short course of pDNA/PEI treatment prevented diabetes onset until experimental endpoints (at 25 weeks of age), while 70-80% of NODf mice in other treatment groups had developed diabetes at this time. In addition, 50% of mice given oral 1MT (and no other treatments) during this experiment developed diabetes faster than control (vehicle-treated) NODf mice. These data indicated that pDNA/PEI treatment was effective in preventing diabetes progression in NODf mice, despite the potential risk of accelerating diabetes onset due to sustained increase in pro-inflammatory cytokine levels (due to the presence of TLR9 ligands in pDNA/PEI nanoparticles). Moreover, IDO slowed diabetes progression in a significant proportion (50%) of NODf mice, indicating the IDO naturally inhibits diabetes progression in mice prone to developing diabetes.

FIG. 10A is a bar graph of serum IFNαβ (U/ml) from B6 (WT) and from mice lacking intact STimulator of INterferon Genes (STING KO mice) treated for 24 hrs. with DNPs containing (CpG+) or lacking (CpG^{free}) TLR9 ligands in cargo DNA. FIG. 10B is a bar graph of IDO activity (pmol/hr/mg) of B6 (WT) and STING (KO) mice treated for 24 hrs. with DNPs containing (CpG+) or lacking (CpG^{free}) TLR9 ligands in cargo DNA.

FIG. 11 is a bar graph of IFNβ1 gene transcripts (wrt to β-actin) from B6 (solid rectangles) or STING-KO mice (white rectangles) were treated with DNPs (i/v, no TLR9 ligands). After 3 hrs spleen cells were stained with CD11c and CD11b (a monocyte marker) mAbs and sorted in a flow cytometer (FACS). Sorted cells were used to prepare RNA for quantitative RT-PCR analysis to detect IFNβ1 and β-actin gene transcripts. Data shows relative levels of IFNβ1 transcripts normalized to β-actin levels in each sorted cell type.

FIG. 12A is a bar graph of T cell proliferative responses (measured as thymidine incorporation) stimulated by dendritic cells from WT (B6) mice treated for 24 hrs. with DNA nanoparticles lacking TLR9 ligands in the absence (black bars) or presence (white bars) of the IDO inhibitor D-1MT. FIG. 12B is a bar graph showing T cell proliferative responses (measured as thymidine incorporation) stimulated by dendritic cells from STING-KO mice treated for 24 hrs. with DNA nanoparticles lacking TLR9 ligands in the absence (black bars) or presence (white bars) of the IDO inhibitor D-1MT). FIG. 12C is a graph of counts per minute versus the number of Tregs for B6 (solid circles) or STING-KO Tregs (open circles).

FIG. 13 is a graph showing that splenic CD11b+DCs express IFNβ1 after DNA nanoparticle treatment. B6 or STING-KO mice were treated with PEI/CpG^{free} nanoparticles for 3 hrs. Splenocytes were stained to detect CD11c and CD11b and sorted. RNA samples from unsorted and FACS-sorted cells were subjected to quantitative RT-PCR analyses to detect IFNβ1 and β-actin transcripts. Data shows IFNβ1:β-actin transcript ratios for unsorted and sorted cell populations, and are representative of 3 and 2 separate experiments using B6 mice and STING-KO mice, respectively.

FIGS. 14A and 14B are bar graphs showing that DNA nanoparticles composed of PEI (FIG. 14A) or biodegradable β amino ester (C32) polymers (FIG. 14B) induced regulatory phenotypes in splenic dendritic cells with comparable efficiencies in mice.

FIG. 15A depicts an experimental procedure to assess the immune modulatory effects of administering DNPs on ovalbumin (OVA)-specific T cell responses (by T cells from OT-1 transgenic mice that recognize OVA) elicited in mice immunized from spleen cells from Act-mOVA transgenic mice expressing OVA. FIG. 15B is a bar graph showing the number of effector (killer, GranzymeB+) OT-1 T cells present in lymph nodes draining (dLNs) sites of OVA immunization in a series of mice treated with DNA nanoparticles containing PEI or three different variants of biodegradable C32 β amino ester polymers (C32-117, C32-118, C32-122) complexed with DNA lacking TLR9 ligands. The number of OT-1 effector T cells present in dLNs of mice treated with vehicle (Vh) is also shown (black bar)

### DETAILED DESCRIPTION OF THE INVENTION

### I. Definitions

The term “pharmaceutically acceptable” means a non-toxic material that does not interfere with the effectiveness of the biological activity of the active ingredients.

The term “pharmaceutically-acceptable carrier” means one or more compatible solid or liquid fillers, dilutants or encapsulating substances which are suitable for administration to a human or other vertebrate animal. (technically—PEI and DNA might both be viewed as ‘active ingredients’ as PEI facilitates DNA entry into cells and rapid release of DNA from endosomes while DNA, once released, triggers downstream responses that affect immune cell functions)—i.e., neither component alone is effective.

The term “carrier” refers to an organic or inorganic ingredient, natural or synthetic, with which the active ingredient is combined to facilitate the application.

The term “effective amount” or “therapeutically effective amount” means a dosage sufficient to provide treatment of the autoimmune or inflammatory disorder, disease, or condition being treated, to induce immune tolerance, or to otherwise provide a desired pharmacologic and/or physiologic effect. The precise dosage will vary according to a variety of factors such as subject-dependent variables (e.g., age, immune system health, etc.), the disease, the disease stage, and the treatment being effected.

The terms “individual,” “individual,” “subject,” and “patient” are used interchangeably herein, and refer to a mammal, including, but not limited to, humans, rodents, such as mice and rats, and other laboratory animals.

The terms “oligonucleotide” or a “polynucleotide” are synthetic or isolated nucleic acid polymers including a plurality of nucleotide subunits of no particular sequence unless otherwise specified.

The term “immunostimulatory polynucleotide” refers to a polynucleotide that serves as a ligand for a pattern recognition receptor (PRR).

The term “immune cell” refers to cells of the innate and acquired immune system including neutrophils, eosinophils, basophils, monocytes, macrophages, dendritic cells, lymphocytes including B cells, T cells, and natural killer cells.

The term “immune-mediated tissue destruction” refers to an injurious immune response.

The term “IDO-dependent regulatory phenotypes” refers to immune suppressive phenotypes that can be induced by stimulating expression of indoleamine 2,3 dioxygenase (IDO) enzyme activity in cells (as in DCs), or as an indirect effect of IDO-expressing DCs (as in IDO-activated Tregs).

The term “complex to” refers to a formation of molecular entity by association involving two or more component molecular entities (ionic or uncharged), or the corresponding chemical species. The bonding between the components can also be covalent.

The term “polynucleotide” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

The term “stimulate expression of” means to affect expression of, for example induction of expression or activity, or induction of increased/greater expression or activity.

The term “CpG sites” or “CpG motifs” refers to regions of a polynucleotide where a cytosine nucleotide occurs next to a guanine nucleotide in the linear sequence of bases along its length. Typically, the cytosine and guanine are separated by only one phosphate. The term “CpG” distinguishes a linear sequence from the CG base-pairing of cytosine and guanine.

The term “TLR9 ligand” refers to a polynucleotide that binds to Toll-like Receptor 9 and induces or activates TLR9 signaling.

The term “suppressive immune response” refers to responses that reduce or prevent the activation or efficiency of innate or adaptive immunity.

The term “IDO-competent cell or cells” as used herein refers to cells that can be induced to express functional indoleamine 2,3-dioxygenase (IDO) enzyme in response to inflammatory signals.

The term “immune tolerance” as used herein refers to any mechanism by which a potentially injurious immune response is prevented, suppressed, or shifted to a non-injurious immune response (Bach, et al., N Eng. J. Med., 347:911-920 (2002)).

The term “tolerizing vaccine” as used herein is typically an antigen-specific therapy used to attenuate autoreactive T and/or B cell responses, while leaving global immune function intact.

### II. Methods and Compositions for Inducing a Suppressive Immune Response

Methods and compositions for inducing or perpetuating a suppressive immune response are disclosed. Suppressive immune responses include, but are not limited to, reducing or inhibiting the secretion of proinflammatory molecules from cells, reducing or inhibiting differentiation, activation or proliferation of effector immune cells such as effector T cells, inducing apoptosis of effector immune cells, increasing or enhancing secretion of immunosuppressive molecules from cells, increasing or enhancing differentiation, activation, recruitment or proliferation of regulatory immune cells, such as Tregs, and/or protecting healthy tissues from immune-mediated attack. For example, inducing or perpetuating an suppressive immune response can include administering an effective amount of the composition to inhibit or reduce the biological activity of an effector T cell or to reduce the amounts of proinflammatory cytokines or other molecules associated with or that promote inflammation at a site of inflammation or autoimmunity. Exemplary proinflammatory molecules include, but are not limited to IL-1β, TNF-α, TGF-beta, IFNγ, IL-17, IL-6, IL-23, IL-22, IL-21, and MMPs.

Compositions including a polynucleotide combined with a vehicle that induces a suppressive immune response in a subject are also disclosed. Typically, the compositions induce an increase in expression of indoleamine 2,3 dioxygenase (IDO) enzyme activity in cells, preferably immune cells.

The disclosed methods and compositions for inducing a suppressive immune response can be used to induce or perpetuate immunosuppression or immune tolerance in a subject in need thereof. In some embodiments the methods are used to induce or promote immune tolerance to known self antigens (“self tolerance”). The methods and compositions can be used, for example, to treat, inflammatory responses, autoimmune diseases, reducing or inhibiting transplant rejection, reducing or preventing graft versus host disease, increasing the effectiveness of tolerizing vaccines and mucosal tolerance, to suppress allergies, and to treat asthma. In some embodiments, the methods for inducing a suppressive immune response in a subject including stimulating expression of indoleamine 2,3 dioxygenase (IDO) enzyme activity in cells of the subject relative to a control to induce the suppressive immune response in the subject. The methods of modulating IDO include inducing cells to acquire IDO-dependent regulatory phenotypes such as inducing IDO-competent cells to express IDO, and inducing regulatory immune cells such as Tregs to acquire one or more suppressive functions either in vivo, or ex vivo are also disclosed.

In some embodiments, the disclosed methods and compositions induce beneficial suppressive immune responses with reduced undesirable toxicities. An exemplary undesirable side effect includes, but is not limited to, a systemic inflammatory responses, typified by rapid and massive release of pro-inflammatory cytokines. In one embodiment, a systemic inflammatory response is characterized by elevated global or systemic levels of IFNγ. In another embodiment, a systemic inflammatory response is characterized by activation of natural killer (NK) cells.

Typically, compositions that induce suppressive immune responses with reduced toxicity lack ligands that bind to pattern recognition receptors for nucleic acids. The receptors can be cell surface receptors or intracellular receptors. The ligands can be absent or masked. Exemplary receptors that bind nucleic acids include, but are not limited to toll-like receptors (TLR3, TLR7, and TLR9). Exemplary ligands that bind toll-like receptors include un-methylated CpG motifs. In some embodiments, the compositions are effective to activate IDO-dependent immune modulation without increasing global or systemic levels of IFNγ and other pro-inflammatory cytokines. In some embodiments a suppressive immune response is induced in a subject by stimulating expression of indoleamine 2,3 dioxygenase (IDO) enzyme activity in cells to induce the suppressive immune response in the subject wherein the induced expression of IDO enzyme activity is not dependent of ligation of TLR9.

A. Polynucleotide Component

Compositions useful for inducing a suppressive immune response include a polynucleotide combined with a vehicle, for example a carrier.

The polynucleotide can be a tolerogenic polynucleotide. A tolerogenic polynucleotide is a polynucleotide that when combined with a vehicle and administered to a subject induces an immune suppressive response, such as immune tolerance. In preferred embodiments the polynucleotide/vehicle complex increases expression of IDO in IDO-competent cells and, as a consequence, induces functionally quiescent Tregs to acquire stable regulatory phenotypes in vivo or ex vivo. The polynucleotides can be single-stranded, double-stranded, circular, partially supercoiled (for example pDNA), and linear (for example pAT), dsDNA, or branched. The polynucleotide can be of prokaryotic or eukaryotic origin. The polynucleotide can be heterologous or autologous. For example, the polynucleotide can be self, or not self. In one embodiment the polynucleotide is bacterial DNA, for example a bacterial plasmid (also referred to herein as “pDNA”). In another embodiment the polynucleotide is salmon sperm DNA, or a fragment thereof.

The polynucleotide can be a plasmid, or an expression vector. In some embodiments the sequence of the polynucleotide includes a coding sequence, for example a sequence encoding a protein, preferably a polypeptide having IDO enzymatic activity. In other embodiments the polynucleotide (also referred to as a nucleic acid) is or encodes an inhibitory nucleic acid such an antisense oligonucleotide, siRNA, RNAi, or miRNA. In other embodiments, the polynucleotide lacks a coding sequence. In still other embodiments, the polynucleotide lacks a coding sequence and is optionally also not an inhibitory nucleic acid. In some embodiments, the polynucleotide is a non-coding, non-inhibitory polynucleotide for example a polyA polynucleotide.

The polynucleotide can be at least 10 nucleotides to at least 50 nucleotides or more in length, including each integral number of nucleotides between 10 and 50. In some embodiments, the polynucleotides can be >3,000 bp (nucleotides).

In other embodiments the polynucleotide is a DNA polynucleotide, however, other types of polynucleotides are also contemplated, including RNA. The nucleotide subunits of the polynucleotide are connected by an internucleotide bond that refers to a chemical linkage between two nucleoside moieties, such as the phosphodiester linkage in nucleic acids found in nature, or linkages well known from the art of synthesis of nucleic acids and nucleic acid analogues. An internucleotide bond may include a phospho or phosphite group, and may include linkages where one or more oxygen atoms of the phospho or phosphite group are either modified with a substituent or replaced with another atom, e.g., a sulfur atom, or the nitrogen atom of a mono- or di-alkyl amino group, such as phosphite, phosphonate, H-phosphonate, phosphoramidate, phosphorothioate, and/or phosphorodithioate linkages. Polynucleotides containing phosphorothioate internucleotide linkages have been shown to be more stable in vivo.

Modified internucleotide linkages also include phosphate analogs, analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. In another embodiment, the polynucleotides are peptide nucleic acids (PNAs), synthetic DNA mimics in which the phosphate backbone of the polynucleotide is replaced in its entirety by repeating N-(2-aminoethyl)-glycine units and phosphodiester bonds are typically replaced by peptide bonds. Each PNA nucleotide typically comprises a heterocyclic base (nucleic acid base), a sugar moiety attached to the heterocyclic base, and a N-(2-aminoethyl)-glycine. The various heterocyclic bases are linked to the backbone by methylene carbonyl bonds, which allow them to form PNA-DNA or PNA-RNA duplexes via Watson-Crick base pairing with high affinity and sequence-specificity. PNAs maintain spacing of heterocyclic bases that is similar to conventional DNA polynucleotides, but are achiral and neutrally charged molecules.

In some embodiments, the polynucleotide is RNA, or an RNA-DNA hybrid. In another embodiment, the polynucleotide is composed of locked nucleic acids (LNA), which are modified RNA nucleotides (see, for example, Braasch, et al., Chem. Biol., 8(1):1-7 (2001)).

In still other embodiments the polynucleotide is constructed with conventional deoxyribose (or ribose) sugars and conventional stereoisomers, but can also be constructed with other sugars, including L enantiomers and alpha anomers. The sugar moiety of the polynucleotides can also be a sugar analog, or include modifications on the sugar moiety, e.g., wherein one or more of the hydroxyl groups are replaced with halogen atoms or aliphatic groups, or are functionalized as ethers, amines, or the like. Sugar moiety modifications include, but are not limited to, 2′-.beta.-aminoetoxy, 2′-O-amonioethyl (2′-OAE), 2′-O-methoxy, 2′-O-methyl, 2-guanidoethyl (2′-OGE), 2′-O,4′-C-methylene (LNA), 2′-O-(methoxyethyl) (2′-OME) and 2′-O-(N-(methyl)acetamido) (2′-OMA).

1. Polynucleotides with Reduced IFNγ-Inducing Attributes

It has been discovered that the IDO pathway can be stimulated in cells without also stimulating potentially toxic side effects. In particular, it has been discovered that IDO expression in cells of a subject can be stimulated without evoking systemic IFNγ production. Therefore, in preferred embodiments, the polynucleotide induces a suppressive immune response compared to a control, without increasing systemic or global levels of IFNγ and optionally without activating natural killer cells to secrete IFNγ. IFNγ is a dimerized soluble cytokine that is the only member of the type II class of interferons. In humans, the IFNγ protein is encoded by the IFNG gene. Genomic, mRNA, and protein sequences are known in the art and can be found, for example, at NCBI Gene ID 3458. IFNγ is an important cytokine for innate and adaptive immunity against viral and intracellular bacterial infections and for tumor control, however, aberrant IFNγ expression is associated with a number of autoinflammatory and autoimmune diseases. Overexpression of IFNγ has been associated with a “cytokine storm” (also known as hypercytokinemia), which is a potentially fatal immune reaction caused by a positive feedback loop between cytokines and immune cells. Natural killer cells (or NK cells) are a type of cytotoxic lymphocyte that constitutes a major component of the innate immune system. Natural killer cells can be induced to secrete IFNγ, resulting in a systemic or global increase in IFNγ in a subject.

2. Reduced Immunostimulatory Elements

It has been discovered that polynucleotides in which immunostimulatory elements are reduced, absent, or masked can induce expression of IDO without stimulating systemic IFNγ production.

Immunostimulatory nucleic acids can serve as ligands for a pattern recognition receptors (PRRs). Examples of PRRs are the Toll-like family of signaling molecules that play a role in the initiation of innate immune responses and also influence the later and more antigen specific adaptive immune responses. In preferred embodiments, the polynucleotides do not serve as a ligand for a Toll-like family signaling molecule. An example of a Toll-like family signaling molecule is a Toll-Like Receptor 9 (TLR9). Therefore, in some embodiments, the polynucleotides are polynucleotides that do not serve as a ligand for TLR9 and/or do not activate TLR9 or TLR9-mediated signaling.

Nucleic acid ligands for PRRs such as TLR9 are typically characterized by a sequence that includes one or more unmethylated CpG motifs, typically interior CpG motifs. Cytosines in CpG dinucleotides can be methylated to form 5-methylcytosine. In mammals, 70% to 80% of CpG cytosines are methylated (Jabbari and Bernardi, Gene, 333:143-9 (2004). Unmethylated CpG sites can be detected by TLR9 on plasmacytoid dendritic cells and B cells in humans (Zaida, et al., Infection and Immunity, 76(5):2123-2129, (2008)). This pathway can be used by cells to detect intracellular viral, fungal, and bacterial pathogen DNA.

Therefore, in some embodiments, the polynucleotides have few, or no interior unmethylated CpG motifs. In other embodiments, the polynucleotides have few, or no unmethylated CpG motifs. In still other embodiments, most or all of the CpG motifs in the polynucleotide are methylated. Polynucleotides without unmethylated CpG motifs are also referred to herein as CpG-free polynucleotides. In some embodiments unmethylated CpG motifs and/or all CpG motifs are masked.

Example of polynucleotides that lack CpG motifs include poly T, poly A, poly G, poly C, poly AT, poly AC, poly AG, and poly GT. The polynucleotides can be single stand or double stranded. For example, in one embodiment a tolerogenic polynucleotide is double stranded poly A:T DNA, also referred to herein as poly dA:T. poly dA:T can be double-stranded and poly dA sequences can be hybridized to poly dT sequences

Other PRR Toll-like receptor include (TLR)3, and TLR7 which may recognize double-stranded RNA, single-stranded and short double-stranded RNAs, respectively, and retinoic acid-inducible gene I (RIG-I)-like receptors, namely RIG-I and melanoma differentiation-associated gene 5 (MDA5), which are best known as RNA-sensing receptors in the cytosol. Therefore, in some embodiments, the polynucleotide does not contain a ligand or does not contain a functional ligand for TLR3, TLR7, or RIG-I-like receptors, or combinations thereof.

In some embodiments, the polynucleotides do not contain ligands for cell surface nucleic acid receptors or contains ligands for cell surface receptors that have been treated to be inoperable, but are able to bind to intracellular receptors. For example, in some embodiments the polynucleotide avoids a cell surface PRR, but is detected by an intracellular nucleic acid receptor. The tolerogenic polynucleotide can avoid cell surface PRR, for example, because ligands for the PRR are masked or absent on the polynucleotide. Typically, the polynucleotides that do not bind cell surface PRR, but do bind intracellular receptors induce immune tolerance, for example by inducing expression of IDO, without stimulating systemic IFNγ production.

In some embodiments immune tolerance is induced when the dose and stability of the polynucleotide delivered inside the cell and released from endosomal vesicles reaches a level that is detected by cytoplasmic DNA sensing mechanisms.

In preferred embodiments, the polynucleotide is non-immunostimulatory. Non-immunostimulatory polynucleotides include polynucleotides that do not serve as a ligand for a PRR, do not contain a ligand for a PRR, or do not contain a functional ligand for PRR.

B. Vehicles

The compositions disclosed herein include a polynucleotide combined with a vehicle. The vehicle can be biologically inert or can induce, promote, or inhibit a biological response. The vehicle or carrier can be non-immunogenic, non-immunostimulatory, non-inflammatory, or combinations thereof. In some embodiments the carrier is not pro-inflammatory. The polynucleotide can be linked to the surface or encapsulated or otherwise loaded into the vehicle, on the vehicle, mixed with the vehicle, or otherwise associated with the vehicle.

Suitable vehicles for use with polynucleotides in the methods described herein, include vehicles that when combined with a polynucleotide and injected into individuals inhibits immune cell activation and effector functions to protect healthy tissues. The vehicle should be compatible with binding stably to nucleic acids, for example is cationic. In some embodiments, the vehicle is capable to delivering the polynucleotide to the interior of a cell. Preferred vehicles include PEI isoforms that can be linear or branched and have variable mean lengths and nanoparticle sizes.

1. Cationic Polymers

In some embodiments, the vehicle is a cationic polymer. Exemplary cationic polymers include polyethylenimine (PEI), polylysine (PLL), polyarginine (PLA), polyvinylpyrrolidone (PVP), chitosan, protamine, polyphosphates, polyphosphoesters, poly(N-isopropylacrylamide), etc. (see for example, U.S. Patent Application No. 20080213377 and U.S. Pat. No. 6,852,709). The polymers can include primary amine groups, imine groups, guanidine groups, and/or imidazole groups. Some examples include poly(beta-amino ester) (PAE) polymers (such as those described in U.S. Pat. No. 6,998,115 and U.S. Pat. No. 7,427,394), which have the additional advantage of being bio-degradable (Lynn, et al., (2000). J Am Chem. Soc. 122: 10761-10768; Lynn, et al., (2001). J Am Chem Soc 123: 8155-8156; Akinc, et al., (2003). Bioconjug Chem 14: 979-988; Anderson, et al., (2005). Mol Ther. 11: 426-434).

The cationic polymer can be unbranched, branched, linear, non-linear, or a combination thereof. Blends, copolymers, and modified cationic polymers can be used. In the some embodiments, the cationic polymer is a linear cationic polymer. The cationic polymer can have a molecular weight between about 0.1 kD and about 250,000 kD, or between about 0.1 kD and about 10,000 kD, or between about 1 kD and 5,000 kD, or between about 50 kD and 1000 kD. The vehicle is preferably greater than 3,000 nucleotides.

The cationic polymer can be suitable for cellular transfection of nucleic acids, such as those discussed in He, et al. Int. J. Pharm., 386(1-2):232-42 (2010) and U.S. Pat. No. 6,013,240. Preferably, the cationic polymer includes polyethylenimine (PEI). The PEI can be deacylated. For example, residual N-acyl moieties can be removed from commercially available PEI, or PEI can be synthesized, e.g., by acid-catalyzed hydrolysis of poly(2-ethyl-2-oxazoline), to yield the pure polycations. An example of an unbranched linear PEI is in vivo-jetPEI™.

2. Particles

The vehicle can be used to form a particle such as a microparticle or a nanoparticle. Nanoparticles generally refers to particles in the range of between 500 nm to less than 0.5 nm, or between 50 and 500 nm, or between 50 and 300 nm. Cellular internalization of polymeric particles is highly dependent upon their size, with nanoparticulate polymeric particles being internalized by cells with much higher efficiency than microparticulate polymeric particles. For example, Desai, et al. have demonstrated that about 2.5 times more nanoparticles that are 100 nm in diameter are taken up by cultured Caco-2 cells as compared to microparticles having a diameter of 1 μm (Desai, et al., Pharm. Res., 14:1568-73 (1997)). Nanoparticles also have a greater ability to diffuse deeper into tissues in vivo than particles of other sizes. In some embodiments the nanoparticles are small enough to diffuse within tissues and enter cells by endocytosis. The nanoparticles used in the methods disclosed herein can be between about 1 nm and 150 nm, or between about 25 nm and 100 nm, or about 50 nm.

Cationic nanoparticles can be constructed at various N/P ratios, which refers to the ratios of moles of the amine groups of cationic polymers to those of the phosphate ones of the polynucleotide. Methods of preparing nanoparticles at a desired N/P ratio are known in the art. See for example Zhao, et al., Biol. Pharm. Bull., 32(4):706-710 (2009) and Ulasov, et al., Mol. Ther., 19(1):103-112 (2011), which describes preparation of PEI nanoparticles at different N/P ratios and determining the effect of N/P on transfection efficiency and toxicity. For example, the N/P ratio for nanoparticles for use with the methods disclosed herein can be between about 3:1 and 12:1, or between about 8:1 and 11:1, or between about 10:1.

In certain embodiments, the particles contain poly beta amino ester.

3. Conjugates

It may also be desirable to attach functional moieties to the polynucleotide or the vehicle. Examples of functional moieties include, but are not limited to, cell penetrating peptides, cell targeting moieties, imaging moieties, chelating moieties, and therapeutic moieties such as synthetic peptides containing epitopes recognized by T cells when presented by APCs on MHC molecules. The attachment can be covalent, or non-covalent. The addition of functional moieties can be used to increase transfection efficiency, target cell specificity, and/or therapeutic index.

Preferred targeting domains target the complex to areas of inflammation or transplantation, or to the spleen or lymph nodes, though any cell or tissue can be targeted. Exemplary targeting domains are antibodies, or antigen binding fragments thereof or another binding partner specific for a polypeptide displayed on the surface of cells that are specific for the desire target cell or tissue. For example, in some embodiments the complexes including a polynucleotide and a carrier are targeted to IDO-competent cells such as those described below.

Alternatively, lymphoid tissue specific targeting can be achieved using lymphoid tissue-specific transcriptional regulatory elements (TREs) such as a B lymphocyte-, T lymphocyte-, or dendritic cell-specific TRE. Lymphoid tissue specific TREs are known in the art.

As discussed above, preferably, the vehicle is a cationic polymer, for example a cationic nanoparticle. Cationic nanoparticles are advantageous for transfection of nucleic acids, in part because nanoparticles displaying a positively charged surface generally exhibit better association and internalization rates with the negatively charged cellular surface (Hillaireau, et al., Cell. Mol. Life. Sci., 66:2873-96 (2009)). In some embodiments, it is desirable to attenuate the cationic surface charge of the nanoparticles to extend the time period nanoparticles can remain in circulation in vivo. Extended circulation can result in a higher dose of nanoparticles reaching a target tissue.

Suitable conjugates and methods for coating or covalently attaching them to cationic polymers are known in the art. See for example (Ulasov, et al., Mol. Ther. 19(1):103-112 (2011), which describes methods of making polynucleotide loaded PEI polyplexes by first activating PEI nanoparticles with commercially available bifunctional polyethylene glycol (PEG), and conjugating a TAT protein transduction oligopeptide to the PEG; or U.S. Patent Application No. 20100323199 which describes methods of PEGylating polymeric nanoparticles. Selection of one or more functional moieties will depend on the target cell type or types and the desired therapeutic result. Examples of functional moieties that can be conjugated to nanoparticles, and methods for attaching them are described in art, see for example U.S. Patent Application No. 20100323199 and U.S. Patent Application No. 20110008457.

4. Formulations

a. Pharmaceutically Acceptable Carriers

The polynucleotide can be combined with a pharmaceutically acceptable vehicle or carrier. The combination can be administered in combination with a second physiologically or pharmaceutically acceptable carrier, excipient, or stabilizer. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In some embodiments, administration is by injection. Typical formulations for injection include a carrier such as sterile saline or a phosphate buffered saline. Viscosity modifying agents and preservatives are also frequently added.

Optional pharmaceutically acceptable excipients especially for enteral, topical and mucosal administration, include, but are not limited to, diluents, binders, lubricants, disintegrants, colorants, stabilizers, and surfactants. Diluents, also referred to as “fillers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE® XL from GAF Chemical Corp).

Stabilizers are used to inhibit or retard decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl mono isopropanolamide, and polyoxyethylene hydrogenated tallow amide. Examples of amphoteric surfactants include sodium N-dodecyl-b-alanine, sodium N-lauryl-b-iminodipropionate, myristoamphoacetate, lauryl betaine and lauryl sulfobetaine.

If desired, the particles may also contain a minor amount of nontoxic auxiliary substances such as wetting or emulsifying agents, dyes, pH buffering agents, or preservatives.

The polynucleotide combined with a vehicle/carrier may be combined with other agents. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

The polynucleotide complexed with a carrier may be coated. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Rohm Pharma, Darmstadt, Germany), zein, shellac, and polysaccharides. Additionally, the coating material may contain conventional carriers such as plasticizers, pigments, colorants, glidants, stabilization agents, pore formers and surfactants.

5. Combination Therapies

The compositions including a polynucleotide combined with a vehicle/carrier disclosed herein can be used alone or in combination with additional therapeutic agents. The additional therapeutic agents include, but are not limited to, immunosuppressive agents (e.g., antibodies against other lymphocyte surface markers (e.g., CD40, alpha-4 integrin) or against cytokines), other fusion proteins (e.g., CTLA-4-Ig (Orencia®), TNFR-Ig (Enbrel®)), TNF-α blockers such as Enbrel, Remicade, Cimzia and Humira, cyclophosphamide (CTX) (i.e. Endoxan®, Cytoxan®, Neosar®, Procytox®, Revimmune™), methotrexate (MTX) (i.e. Rheumatrex®, Trexall®), belimumab (i.e. Benlysta®), or other immunosuppressive drugs (e.g., cyclosporin A, FK506-like compounds, rapamycin compounds, or steroids), anti-proliferatives, cytotoxic agents, or other compounds that may assist in immunosuppression.

In some embodiments, the additional therapeutic agent functions to inhibit or reduce T cell activation and cytokine production through a separate pathway. In one such embodiment, the additional therapeutic agent is a CTLA-4 fusion protein, such as CTLA-4 Ig (abatacept). CTLA-4 Ig fusion proteins compete with the co-stimulatory receptor, CD28, on T cells for binding to CD80/CD86 (B7-1/B7-2) on antigen presenting cells, and thus function to inhibit T cell activation. In some embodiments, the additional therapeutic agent is a CTLA-4-Ig fusion protein known as belatacept. Belatacept contains two amino acid substuitutions (L104E and A29Y) that markedly increase its avidity to CD86 in vivo. In another embodiment, the additional therapeutic agent is Maxy-4.

In another embodiment, the second therapeutic is a second agent that induces IDO expression. Second therapeutics that induce IDO expression are described in Johnson, et al., Immunotherapy, 1(4):645-661 (2009), and U.S. Pat. Nos. 6,395,876 and 6,451,840. In one embodiment, the second therapeutic that induces IDO expression is a nanoparticle loaded with an expression vector that encodes an IDO1 or IDO2 polypeptide.

In another embodiment, the second therapeutic agent preferentially treats chronic transplant rejection or GvHD, whereby the treatment regimen effectively targets both acute and chronic transplant rejection or GvHD. In another embodiment the second therapeutic is a TNF-α blocker.

In another embodiment, the second therapeutic agent increases the amount of adenosine in the serum, see, for example, WO 08/147,482. In some embodiments, the second therapeutic is CD73-Ig, recombinant CD73, or another agent (e.g. a cytokine or monoclonal antibody or small molecule) that increases the expression of CD73, see for example WO 04/084933. In another embodiment the second therapeutic agent is Interferon-beta.

In some embodiments, the compositions are used in combination or succession with compounds that increase Treg activity or production. Exemplary Treg enhancing agents include but are not limited to glucocorticoid fluticasone, salmeteroal, antibodies to IL-12, IFNγ, and IL-4; vitamin D3, and dexamethasone, and combinations thereof. Antibodies to other proinflammatory molecules can also be used in combination or alternation with the disclosed compositions. For example, antibodies can bind to IL-6, IL-23, IL-22 or IL-21.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art.

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds are known in the art. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

B. Methods of Inducing an Immune Suppressive Response

1. Methods of Treating Diseases and Disorders

a. In Vivo Applications

For in vivo applications, an effective amount of a pharmaceutical composition including a polynucleotide complexed to a vehicle/carrier is administered to a subject. In some embodiments the composition is administered in an amount effective to induce local or systemic induction of Treg suppressor function compared to a control. In some embodiments the composition is administered in an amount effective to increase expression of IDO in IDO-competent cells compared to a control. The compositions can also be administered in an amount effective to inhibit, reduce, or alleviate one or more symptoms of the disease or condition to be treated.

### Routes of Administration

In some embodiments the compositions are administered systemically. In some embodiments, the compositions are administered locally to a site of inflammation or autoimmunity, or to immune tissues or organs, such as lymph nodes or the spleen. Methods of delivering the disclosed compositions include, but are not limited to, oral delivery, nasal inhalation, nebulization, intraperitoneal injection (IP), sub-cutaneous (SC), systemic injection (IV), organ infusion (for donor organs used in transplantation) and topical applications, including, but not limited to, transdermal and transmucosal applications. In some embodiments, the route of administration depends of the organ or tissue to be treated.

In some embodiments, the composition is administered in an aqueous solution, by parenteral injection. The formulation may also be in the form of a suspension or emulsion. In general, pharmaceutical compositions are provided including an effective amount of a composition, and optionally include pharmaceutically acceptable carrier.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

For topical formulations, administration may be most effective when applied to the lungs, nasal, oral (sublingual, buccal), vaginal, or rectal mucosa.

b. Ex Vivo Applications

In some embodiments, the compositions disclosed herein are used to modulate cells ex vivo. In some embodiments, the target cells are isolated from the subject to be treated (autologous cells, tissues or organs) or from an allogenic host. For ex vivo applications, target cells are typically removed from a subject or obtained from another source prior to contacting the cells with the compositions disclosed herein. In some embodiments, the target cells are cells that can be induced to express IDO when contacted with a composition including a polynucleotide combined or complexed to a vehicle or carrier. In one embodiment, the target cells are IDO-competent dendritic cells, for example dendritic cells in mice and humans that display attributes of plasmacytoid DCs (pDCs). The cells can also be hematopoietic progenitor or stem cells that are induced to form IDO-competent cells in culture (Munn, D. H., et al., Science 297:1867-1870 (2002)).

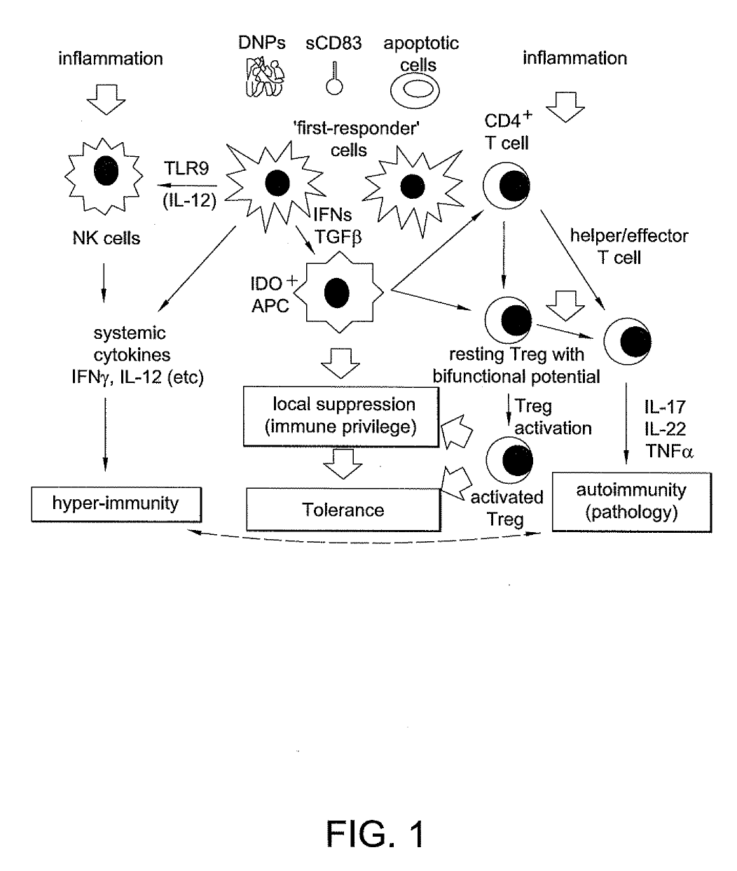

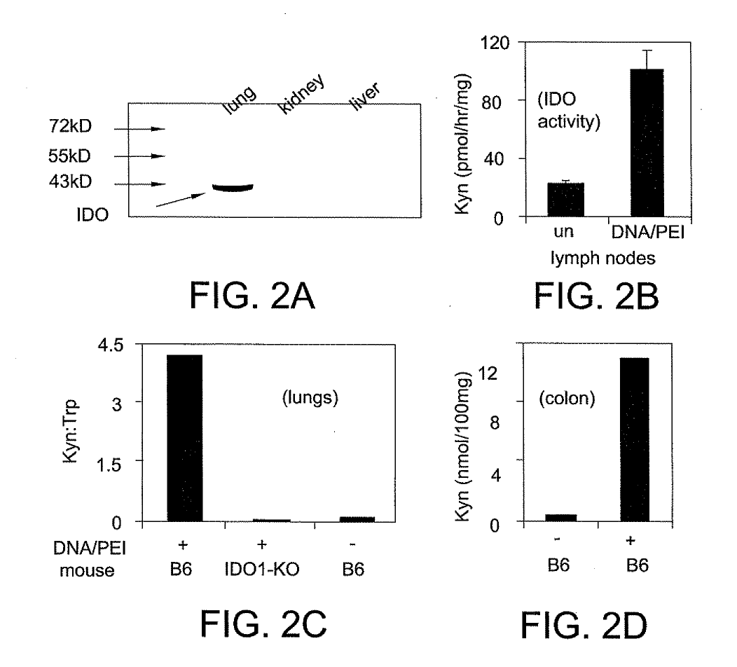

In some embodiments phagocytic myeloid DCs or MDs such as DC2.4 cells or physiological first responder phagocytes are co-cultured with IDO-competent dendritic cells. In the model shown in FIG. 1 phagocytic myeloid DCs and/or physiological first responder phagocyte cells engulf polynucleotide loaded nanoparticles, sense the polynucleotide, and elicit rapid release of IFNs that induce IDO-competent dendritic cells to express IDO. In some embodiments the phagocytic myeloid DCs or physiological first responder phagocytes detect DNA by a TLR9-independent pathway and induce expression of IDO enzyme activity in competent dendritic cells with little or no changes in global or systemic levels of IFNγ.

In some embodiments IDO-competent cells are induced to expression IDO by interferon type I. In some embodiments, interferon type I is added directly to the culture. In some embodiments interferon type I is not required for the composition to induce an immune suppressive response or stimulate expression of IDO.

Target cells can be isolated and enriched by one of skill in the art. For example, cells can be selected by positive and negative selection techniques. Cells can be selected using commercially available antibodies which bind to surface antigens, e.g. CD19, using methods known to those of skill in the art. For example, the antibodies may be conjugated to magnetic beads and immunogenic procedures utilized to recover the desired cell type. Other techniques involve the use of fluorescence activated cell sorting (FACS).

After the target cells are contacted with a composition that stimulates expression of IDO, the cells can be introduced into the subject, increasing the number of cells with IDO enzyme activity in the subject.

In another embodiment, cells contacted with a composition including a polynucleotide combined with a vehicle ex vivo are used to modulate a second target cell type. For example, cells with increased IDO enzyme activity ex vivo can be used to induce naïve CD4 T cells to differentiate into Foxp3-lineage Tregs and/or induce suppressor function in Tregs ex vivo. After the second target cell type has been modulated by the IDO-competent cells ex vivo, the second target cell type can be introduced into the subject, increasing the number of immune suppressive cells in the subject.

In some embodiments, cells modulated ex vivo are introduced into the subject at a site of inflammation, autoimmune disease, transplantation, or another site in need of immune tolerance. In some embodiments, cells modulated ex vivo are administered to immune tissues or organs, such as lymph nodes or the spleen.

2. Diseases to be Treated

The compositions and methods disclosed herein can be used to inhibit immune-mediated tissue destruction for example in a setting of inflammatory responses, autoimmune and allergic diseases, and transplant rejection.

a. Inflammatory and Autoimmune Disorders

In certain embodiments, the disclosed compositions and methods for inducing or perpetuating a suppressive immune response are used to treat an inflammatory response or autoimmune disorder in a subject. For example, the disclosed methods can be used to prophylactically or therapeutically inhibit, reduce, alleviate, or permanently reverse one or more symptoms of an inflammatory response or autoimmune disorder. An inflammatory response or autoimmune disorder can be inhibited or reduced in a subject by administering to the subject an effective amount of a composition including a polynucleotide combined with a vehicle in vivo, or cells modulated by a polynucleotide combined with a vehicle ex vivo as described above.

Representative inflammatory responses and autoimmune diseases that can be inhibited or treated include, but are not limited to, rheumatoid arthritis, systemic lupus erythematosus, alopecia greata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

(i) Rheumatoid Arthritis

In a one preferred embodiment, polynucleotides combined with a vehicle/carrier are used to reduce, inhibit, alleviate or permanently reverse one or more symptoms of rheumatoid arthritis. Rheumatoid arthritis can be inhibited or reduced in a subject by administering to the subject an effective amount of a composition including a polynucleotide combined with a vehicle in vivo, or cells modulated by a polynucleotide combined with a vehicle ex vivo as described above. For example, treatment of arthritis with polynucleotide complexed with a carrier can reduce joint swelling, reduce infiltration of leukocytes such as neutrophils into the synovial membrane/joint space or surrounding tissue (i.e., joint inflammation), reduce synovial lining layer hyperplasia; and reduce pannus formation and necrosis/erosion of cartilage (as a measure of joint destruction) reduce levels of IL-17 and IL-6, or other pro-inflammatory cytokines by cells from inflamed inguinal and popliteal lymph nodes draining sites of joint inflammation. Thus, polynucleotides combined with a vehicle can be used to attenuate innate and adaptive immunity that drives joint destruction.

(ii) Type I Diabetes

In another preferred embodiment, polynucleotides combined with a vehicle are used prophylactically or therapeutically to reduce, inhibit, alleviate or permanently reverse one or more symptoms of type I diabetes. Type I diabetes can be inhibited or reduced in a subject by administering to the subject an effective amount of a composition including polynucleotides combined with a vehicle in vivo, or cells modulated by a polynucleotide combined with a vehicle vivo as described above. Preferably, the compositions are administered in an effective amount to reduce or inhibit destruction of insulin producing cells and tissue by the subject's immune system.

Type 1 diabetes (also known as Diabetes mellitus type 1, IDDM, or, formerly, juvenile diabetes) is a form of diabetes mellitus that results from autoimmune destruction of insulin-producing beta cells of the pancreas. The subsequent lack of insulin leads to increased blood and urine glucose. Symptoms to type I diabetes include, but are not limited to, polyuria (frequent urination), polydipsia (increased thirst), polyphagia (increased hunger), and weight loss. Treatment with a polynucleotide combined with a vehicle may also be effective to reduce molecular symptoms of type I diabetes, including, but not limited to destruction of beta cells, and/or autoimmune responses towards beta cells, including, but not limited to expansion of autoreactive CD4+ and CD8+ T helper cells, autoantibody-producing B cells and activation of the innate immune system.

b. Transplant Rejection

In another embodiment, the disclosed compositions and methods for inducing or perpetuating a suppressive immune response can be used prophylactically or therapeutically to reduce or inhibit graft rejection or graft verse host disease. Transplant rejection occurs when a transplanted organ or tissue is not accepted by the body of the transplant recipient. Typically rejection occurs because the immune system of the recipient attacks the transplanted organ or tissue. The disclosed methods can be used to promote immune tolerance of the transplant or graft by the receipt by administering to the subject an effective amount of a composition including a polynucleotide combined with a vehicle in vivo, or cells modulated by a polynucleotide combined with a vehicle ex vivo as described above.

i. Transplants

The transplanted material can be cells, tissues, organs, limbs, digits or a portion of the body, for example the human body. The transplants are typically allogenic or xenogenic. The disclosed compositions are administered to a subject in an effective amount to reduce or inhibit transplant rejection. The compositions can be administered systemically or locally by any acceptable route of administration. In some embodiments, the compositions are administered to a site of transplantation prior to, at the time of, or following transplantation. In one embodiment, compositions are administered to a site of transplantation parenterally, such as by subcutaneous injection.

In other embodiments, the compositions are administered directly to cells, tissue or organ to be transplanted ex vivo. In one embodiment, the transplant material is contacted with the compositions prior to transplantation, after transplantion, or both.

In other embodiments, the compositions are administered to immune tissues or organs, such as lymph nodes or the spleen.

The transplant material can also be treated with enzymes or other materials that remove cell surface proteins, carbohydrates, or lipids that are known or suspected of being involved with immune responses such as transplant rejection.

(a). Cells

Populations of any types of cells can be transplanted into a subject. The cells can be homogenous or heterogenous. Heterogeneous means the cell population contains more than one type of cell. Exemplary cells include progenitor cells such as stem cells and pluripotent cells which can be harvested from a donor and transplanted into a subject. The cells are optionally treated prior to transplantation as mention above.

(b). Tissues

Any tissue can be used as a transplant. Exemplary tissues include skin, adipose tissue, cardiovascular tissue such as veins, arteries, capillaries, valves; neural tissue, bone marrow, pulmonary tissue, ocular tissue such as corneas and lens, cartilage, bone, and mucosal tissue. The tissue can be modified as discussed above.

(c). Organs

Exemplary organs that can be used for transplant include, but are not limited to kidney, liver, heart, spleen, bladder, lung, stomach, eye, tongue, pancreas, intestine, etc. The organ to be transplanted can also be modified prior to transplantation as discussed above.

One embodiment provides a method of inhibiting or reducing chronic transplant rejection in a subject by administering an effective amount of the composition to inhibit or reduce chronic transplant rejection relative to a control.

Ii. Graft-Versus-Host Disease (GVHD)

The disclosed compositions and methods can be used to treat graft-versus-host disease (GVHD) by administering an effective amount of the composition to alleviate one or more symptoms associated with GVHD. GVHD is a major complication associated with allogeneic hematopoietic stem cell transplantation in which functional immune cells in the transplanted marrow recognize the recipient as “foreign” and mount an immunologic attack. It can also take place in a blood transfusion under certain circumstances. Symptoms of GVD include skin rash or change in skin color or texture, diarrhea, nausea, abnormal liver function, yellowing of the skin, increased susceptibility to infection, dry, irritated eyes, and sensitive or dry mouth.

In another embodiment, the disclosed compositions and methods for inducing or perpetuating a suppressive immune response can be used prophylactically or therapeutically to suppress allergies and/or asthma and/or inflammation in lungs. Allergies and/or asthma and/or inflammation in the lungs can be suppressed, inhibited or reduced in a subject by administering to the subject an effective amount of a composition including a polynucleotide combined with a vehicle in vivo, or cells modulated by a polynucleotide combined with a vehicle ex vivo as described above.

In some embodiments, the composition induces IDO-competent cells to have increased IDO enzyme activity in the lungs. In one embodiment, the IDO-competent cells are lung epithelial cells.

It has been reported that the induction of pulmonary IDO by immunostimulatory polynucleotides protects the lung from Th2-driven lung inflammation and experimental asthma. Likewise, the induction of IDO in a SCID/Th1 transfer model attenuated Th1-driven lung inflammation. However, in this case, in contrast to the Th2 transfer model, the inhibition of lung inflammation by immunostimulatory polynucleotide administration was buffered by the intrinsic ability of Th1 cells to induce pulmonary IDO activity after OVA challenge, most probably via the production of IFNγ, (Hayashi, et al., J. Clin. Investig., 114(2):270-279 (2004)). Therefore, the compositions can be delivered in an effective amount to induce a level of IDO activity that can inhibit Th-mediated lung inflammation, for example by (a) depleting trp availability in the microenvironment; (b) promoting the generation of various toxic trp metabolites, which induce Th cell death; (c) inducing generation of other compounds, e.g., formylkynurenine, through a reaction that removes oxygen radicals at inflammatory sites; and/or (d) in the case of Th2-mediated lung inflammation, inhibiting the generation of 5-hydroxytryptamine, a potent airway constrictor.

4. Methods of Modulating Vaccines

Tolerogenic vaccines deliver antigens with the purpose of suppressing immune responses and promoting robust long-term antigen-specific immune tolerance. For example, Incomplete Freund's Adjuvant (IFA) mixed with antigenic peptides stimulates Treg proliferation (and/or accumulation) and IFA/Insulin peptide prevents type I diabetes onset in susceptible mice, though this approach is ineffective in reversing early onset type I diabetes (Fousteri, G., et al., 53:1958-1970 (2010)). The compositions and methods disclosed herein are also useful for controlling the immune response to an antigen. For example, a composition including a polynucleotide combined with a vehicle can be used to potentiate the effect of a tolerizing vaccine. In some embodiments, the tolerizing vaccine is a DNA vaccine. DNA immunization provides a non-replicating transcription unit that serves as a template for the synthesis of proteins or protein segments to induce antigen specific immune responses in the host (Ho, et al., Autoimmunity, 39(8):675-682 (2006)). Injection of DNA encoding foreign antigens promotes immunity against a variety of microbes and tumors. In autoimmune diseases DNA vaccines induce tolerance to the DNA-encoded self-antigens. The DNA-encoded self-antigen depends on the disease to be treated, and can be determined by one of skill in the art.

Compositions including a polynucleotide combined with a vehicle can be used to enhance the immune suppressive effect of DNA vaccines designed to induce tolerance to the DNA-encoded self-antigens. The compositions can be administered in combination with or as a component of, a tolerizing vaccine composition. A vaccine typically contains an antigen, or a nucleic acid encoding an antigen as in DNA vaccines, and optionally may include one or more adjuvants. The antigen, for example, a DNA-encoded self-antigen, depends on the disease to be treated, and can be determined by one of skill in the art. A composition including a polynucleotide combined with a vehicle is administered in combination with a vaccine is typically administered in amount effective to increase immunosuppression compared to administration of the vaccine alone.

Suitable adjuvants can be, but are not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

5. Methods of Modulating Mucosal Tolerance

Mucosal tolerance, also referred to as oral tolerance, is the absence of an immune response to an antigen that is exposed through mucosal surfaces such as the gastrointestinal tract, genitoutinary, or bronchial tissue. Mucosal tolerance is a natural immunologic process driven by the presence of an exogenous antigen, whereby external agents (antigens) that gain access to the body via a natural route become part of the self (Iian, Human Immunol., 70:768-776 (2009)). Antigen-specific therapy via mucosal tolerance is a physiologic means to manipulate immune responses, is nontoxic, and can be administered on a chronic basis. When self-antigens are administered, mucosal tolerance can be used to treat inflammatory responses and autoimmune diseases.

Compositions including a polynucleotide combined with a vehicle can be used to enhance mucosal tolerance, particular as it applies to treatment of autoimmune diseases and inflammatory responses. The compositions can be administered in combination with, or as a component of, a mucosal tolerance composition. A mucosal tolerance composition typically contains an antigen, for example a whole protein, a peptide, an altered peptide or a nucleic acid encoding an antigen, and optionally may include one or more adjuvants. Suitable adjuvants are known in the art and discussed above with respect to tolerizing vaccines. The antigen, for example a self-antigen, depends on the disease to be treated, and can be determined by one of skill in the art.

A composition including a polynucleotide combined with a vehicle administered in combination with a mucosal tolerance composition is typically administered in amount effective to increase immunosuppression compared to administration of the mucosal tolerance composition alone. The mucosal composition is typically administered to a mucosa, for example, oral, nasal, and gastrointestinal mucosa. Routes of administration of the antigen include, but are not limited to oral, nasal, and parentetal. A composition including a polynucleotide combined with a vehicle that is administered in combination with a mucosal tolerance composition can be administered via the same route, or a separate route of administration, such as those described above. Mucosal tolerance may be particularly effective for treatment of the autoimmune diseases discussed above, for example, encephalomylelitis, myasthenia gravis, Neuritis, uveoretinitis, insulin dependent diabetes mellitus (type I diabetes), and arthritis (Xiao, et al., Clin. Immunol. Immunopath., 85(2):119-28 (1997)).

C. Methods of Modulating IDO Signaling

As discussed above, the disclosed compositions including a polynucleotide combined with a vehicle induce a suppressive immune response. Typically, the compositions induce an increase in expression of indoleamine 2,3 dioxygenase (IDO) enzyme activity in IDO-competent cells. As a result, the compositions can be used to induce cells to acquire regulatory phenotypes that suppress innate and adaptive T cell responses to defined exogenous antigens and autoantigens. In some embodiments, the methods describe above include an effective amount of the composition to induce an immune regulatory phenotype. IDO signaling, IDO-dependent immune regulatory phenotypes, and methods of using the disclosed compositions to induce immune regulatory phenotypes are described in detail below.

1. IDO Signaling

IDO is an intracellular heme-containing enzyme that catalyzes the initial rate-limiting step in tryptophan degradation along the kynurenine (Kyn) pathway (Mellor, et al., Nat Rev Immunol, 4:762-774 (2004)). Tryptophan starvation by IDO consumption inhibits T-cell activation, whereas products of tryptophan catabolism, such as kynurenine derivatives and O₂ free radicals, regulate T-cell proliferation and survival (Mellor, et al., Immunol Today, 20:469-473 (1999), Mellor, et al., J. Immunol., 168:3771-3776 (2002)). IDO is widely expressed in human tissues and cell subsets and is induced during inflammation by IFNγ and other inflammatory cytokines (Däubener, et al., Adv. Exp. Med. Biol., 467:517-524 (1999). Two closely linked, homologous genes (IDO1 and IDO2) located in syntenic regions of chromosome 8 in humans and mice encode IDO proteins (Ball, et al., Gene, 396(1):2003-213 (2007)), and six introns in IDO-related genes are conserved from humans to mollusks, implying conservation for 600 million years (Suzuki, et al., Gene, 308:89-94 (2003)). All mammalian IDO genes studied to date possess one or more IFN response elements (ISRE and GAS) in their promoter regions, and IFNs produced at local sites of inflammation are potent inducers of IDO in several cell types, such as some DCs, macrophages, eosinophils, epithelial and endothelial cells (Hayashi, et al., J. Clin. Invest., 114(2):270-9 (2004)).

IDO gene expression is induced in some cell types by type I and type II IFNs, and some DC subsets suppress effector T cell responses, activate regulatory (Foxp3+) T cells (Tregs) and block pro-inflammatory cytokine production by other activated immune cells when induced to express IDO (Mellor, A. L. and Munn, D. H., Nat Rev Immunol, 8:74-80 (2008), Mellor, A. L., and Munn, D. H., J Immunol, 186:4535-4540 2011)). By these mechanisms cells expressing IDO at sites of inflammation suppress immune-mediated damage to healthy tissues in settings of autoimmunity, transplantation, and pregnancy, though IDO also promotes persistence of tumors and may contribute to persistence of some clinically important infections such as HIV and leishmania (Munn, D. H., Curr Med Chem, 18:2240-2246 (2011), Makala, et al., J Infect Dis, 203:715-725 (2011), Boasso, et al., J Immunol, 182:4314-4320 (2009)). Thus physiologic IDO activity is a key factor that regulates innate and adaptive immunity at sites of chronic inflammation associated with a range of clinical syndromes.

IDO regulates T cell responses by causing Trp withdrawal and Kyn release. Trp withdrawal stimulates ER stress responses by triggering activation of the ribosome associated protein kinase general control of non-derepressible-2 (GCN2). GCN2 ablation renders Tregs insensitive to the regulatory effects of IDO (Munn, et al., Immunity, 22:10 (2005), Baban, et al., J Immunol, 183:275-2483 (2009), and dendritic cells (DCs) lose the ability to produce IFNα following B7 ligation (Manlapat, et al., Eur J. Immunol, 37:1064-1071 (2007)). GCN2 (encoded by the eIFK4 gene) has also been implicated in halofuginone mediated suppression of TH17 responses (Sundrud, et al., Science, 325:1334-1338 (2009)), and as a key pathway driving potent immune responses to yellow fever vaccine (YF17D) in humans (Querec, et al., Nat Immunol, 10:116-125 (2009)). Thus the GCN2 pathway is essential for immune cells to elaborate responses to critical inflammatory cues, and IDO-mediated Trp withdrawal activates regulatory responses via GCN2.

Trp catabolites (e.g. 3-HAA) released by IDO expressing cells also inhibit destructive TH17 responses in chronically infected lungs, in part by blocking PDK1 signaling that activates NFκB in T cells and activating Tregs (Fallarino, et al., J Immunol, 176:6752-6761 (2006), Hayashi, et al., Proc Natl Acad Sci USA, 104:18619-18624 (2007), Desvignes, L. and Ernest, J. D., Immunity, 31:974-985 (2009), Romani, et al., Nature, 451:211-215 (2008)). Aryl hydrocarbon receptor (AHR) ligands induce IDO in bone marrow derived DCs (BMDCs), and Kyn activates the AHR pathway in naïve CD4 T cells to promote Treg generation (Mezrich, et al., J Immunol, 185:3190-3198 (2010)). Thus IDO may influence immune outcomes via key metabolic pathways responsive to Trp withdrawal and Trp catabolites.

2. Regulatory Phenotypes

a. IDO-Competent DCs and IDO-Dependent Suppression

Traditionally, DCs are considered pivotal in eliciting effector/helper T cell responses as DCs are equipped with arrays of pathogen and damage associated molecular pattern (PAMPs & DAMPs) receptors to detect inflammatory signals, acquire and process antigens, and present antigens to T cells. However DCs are phenotypically diverse and functionally heterogeneous. In certain settings of inflammation some DCs acquire potent regulatory functions that promote and maintain local immune privilege (Mellor, A. L., and Munn, D. H., Nat Rev Immunol, 8:74-80 (2008)). DCs competent to express IDO in mice and humans have been identified (Munn, et al., Science, 297:1867-1870 (2002), Mellor, et al., J. Immunol, 175:5601-5605 (2005), Chen, et al., J Immunol, 181:5396-5404 (2008)). In mice, IDO-competent DCs are a rare, but distinctive DC population that display attributes of plasmacytoid DCs (pDCs) and B cells, such as CD19 expression (Baban, et al., Int Immunol, 17:909-919 (2005), Johnson, et al., Proc Natl Acad Sci USA 107:10644-10648 (2010)). In mice treated with CpGs (TLR9 ligands) CD19+ DCs selectively expressed IDO to activate Tregs, and block T cell proliferation, pro-inflammatory cytokine expression (e.g. IL-6, IL-17) and Treg re-programming (Baban, et al., J Immunol, 183:2475-2483 (2009), Mellor, et al., J Immunol, 175:5601-5605 (2005)). Under IDO-deficient conditions regulatory responses to TLR9 ligation are abrogated. Thus DCs competent to express IDO are pivotal regulators of T cell and Treg responses to inflammation.

b. Treg Functional Status

Naïve CD4+ T cells can differentiate into Foxp3-lineage Tregs or TH17 effector T cells depending on the signals they receive. Tregs must be activated to acquire suppressive phenotypes, but resting Tregs can also undergo functional re-programming to acquire polyfunctional helper/effector phenotypes. Much research has focused on defining conditions that promote Treg differentiation and re-programming; however factors that influence these responses in physiologic settings remain poorly defined. Pre-formed Foxp3+ Tregs can contribute to regulatory or stimulatory responses in defined murine models of infection, tumor growth and skin transplant rejection (Mellor, A. L. and Munn, D. H., J Immunol, 186:4535-4540 (2011)).

It has also been reported that Tregs—not naïve CD4 T cells—were the obligate source of ‘T cell help’ for primary CD8 T cell responses after vaccination (Sharma, et al., Immunity, 33:942-954 (2010)). This finding indicates that resting Tregs serve as a de facto pool of pre-activated, ‘rapid response’ cells that mediate regulation or provide help, contingent on the signals they receive. As polyclonal Tregs recognize ubiquitous self-antigens, access to exogenous antigens is not limiting, enabling Tregs to respond rapidly to inflammatory cues (Mellor, A. L. and Munn, D. H., J Immunol, 186:4535-4540 (2011)). Following CpG treatment to induce CD19+ DCs to express IDO, resting Tregs acquired suppressor functions via IDO; conversely, CpG treatment under conditions of IDO ablation induced resting Tregs to express pro-inflammatory cytokines (Baban, et al., J Immunol, 183:2475-2483 (2009)). Thus IDO-competent DCs serve as pivotal physiologic regulators of Treg functional responses to inflammatory cues such as TLR9 ligands. IDO-mediated Treg activation following CpG treatment was also shown to be dependent on the presence of T cells, TGFβ, and intact CTLA4/B7 and PD-1/PD-L co-stimulatory pathways (Baban, et al., J Immunol, (2011)).

2. Methods of Inducing Immune Regulatory Phenotypes in Cells

Compositions including a polynucleotide combined with a vehicle can be used to modulate or regulate the activity of a cell. In some embodiments the methods include contacting one or more cells with an effective amount of the composition to induce IDO expression. IDO expressing cells include, but are not limited to fibroblasts, dendritic cells, macrophages, and epithelial cells (Hayashi, et al., J. Clinical Invest., 114(2):270-279 (2004)). In some embodiments, the compositions are administered in an effective amount to induce IDO expression in IDO-competent dendritic cells. In some embodiments, IDO-competent dendritic cells display attributes of plasmacytoid DCs (pDCs) and B cells, such CD19 and/or B-lymphoid (Pax5+) lineage cells. In some embodiments IDO-competent cells also co-express CD11c. Induction of IDO expression can be measured as an increase in IDO expression in cells or tissue treated with a composition compared to a control, for example cells or tissue that is not treated with a composition.

In some embodiments, the compositions described herein are used to modulate or regulate the activity of Tregs. For example, a composition that stimulates expression of IDO can be administered to cells or tissue in an effective amount to induce Tregs to acquire or enhance a suppressor function compared to a control, for example the level of Treg suppressor function in the absence of administering the composition to cells or tissue. In some embodiments Tregs are induced to acquire a suppressor function by signaling from an IDO-competent cell. In one embodiment, the IDO-competent cell is an IDO-competent dendritic cell.

Treg suppressor functions include, but are not limited to, reducing or suppressing the proliferation or cytokine secretion of, or inducing apoptosis in one or more effector T cells. For example, Tregs with enhanced suppressor function can have an enhanced suppressive effect on the Th1, Th17, Th22 and/or other cells that secrete, or cause other cells to secrete, inflammatory molecules to reduce the level of IFNγ and/or IL-17 and/or IL-6 produced. Tregs with enhanced suppressor function can also exhibit increased proliferation, and/or increase recruitment to sites of inflammatory, and/or have enhanced production of IL-10, IL-2 and TGF-β, which can suppress the Th1 and/or Th17 pathways. IDO-activated Tregs may also regulate suppressor function via PD-1/PDL-1 and/or CTLA4/B7 signaling. Therefore, in some embodiments the compositions include additional therapeutic agents that induce, activate, perpetuate, or maintain PD-1/PDL-1 and/or CTLA4/B7 signaling.

In some embodiments, the cells are contacted with the composition ex vivo, or the composition is administered in vivo, for example, locally or systemically, and contact the cells or tissue by diffusion as described above.

### III. Methods of Testing Efficacy and Toxicity, and Controls

Polynucleotides combined with a vehicle or complexed with carrier suitable for use in the claimed methods can be identified experimentally using the methods known in the art and the methods and assays disclosed in the Examples below.

For example, induction of IDO can be determined by measuring kynurenine concentration in cell free homogenates prepared from test tissue, for example from an animal model, as described in Hoshi, et al., J. Immunol., 185:3305-3312 (2010). Tissue can be tested before and after treatment with tolerogenic polynucleotide complexed with a carrier. An increase in kynurenine concentration in cell free homogenates is indicative of an increase in IDO expression. Kynurenine concentration can be measured by high performance liquid chromatography (HPLC), and IDO activity can be calculated as pmol of kynurenine generated per hour of reaction per mg tissue. IDO levels can also be determined by standard immuno assay (including but not limited to enzyme-linked immunosorbent assay (ELISA), immunohistochemistry, and flow cytometry) of test cells or tissue. Increased IDO expression can be higher IDO expression after treatment with tolerogenic polynucleotide complexed with a carrier compared to untreated cells or tissue.

IFNγ levels can be determined by standard immuno assay (including but not limited to enzyme-linked immunosorbent assay (ELISA), immunohistochemistry, and flow cytometry) of test tissue or serum. Increased expression can be higher IFNγ levels after treatment with polynucleotide loaded nanoparticles compared to untreated tissue. An increase in kynurenine concentration without increasing global or systemic IFNγ expression following treatment with tolerogenic polynucleotide complexed with a carrier is useful in the methods of immunosuppression with reduced toxicity as described above. This combination is effective at inducing an immune suppressive response or immune tolerance with low or reduced toxicity compared to an immunostimulatory polynucleotide complexed with a carrier.

Suitable controls are known in the art and can be determined based on the disease or disorder to be treated, or the desire therapeutic effect. Examples of controls include, but are not limited to, comparing cells or tissue treated with tolerogenic polynucleotide complexed with a carrier to 1) untreated cells or tissue, 2) cells or tissue treated with the polynucleotide alone, or 3) cell or tissue treated with carrier alone. In some embodiments cells or tissue treated with tolerogenic polynucleotide complexed to a carrier exhibit an increase in expression of IDO, an immunosuppressive response, immune tolerance or combinations thereof, compared to a control, as discussed above. In some embodiments tolerogenic polynucleotide complexed to a carrier inhibit, reduce, or alleviate one or more symptoms of an inflammatory response, or an autoimmune disease, in a subject compared to 1) an untreated subject, 2) a subject treated with the polynucleotide alone, or 3) a subject treated with carrier alone. In some embodiments tolerogenic polynucleotide complexed induce expression of IDO, an immunosuppressive response, immune tolerance or combinations thereof in a subject compared to 1) an untreated subject, 2) a subject treated with the polynucleotide alone, or 3) a subject treated with carrier alone.

In some embodiments, a polynucleotide combined with a vehicle induces an immune suppressive response or immune tolerance with low or reduce toxicity compared to a control. Suitable controls for assaying reduced toxicity are known in the art. Examples of controls include, but are not limited to, comparing cells, tissue, or a subject treated with polynucleotides in which immunostimulatory elements are reduced, absent, or masked complexed with a carrier to cells, tissue, or a subject treated with an immunostimulatory polynucleotide, for example an unmethylated CpG oligomer, complexed with a carrier. When compared to treatment of cells or tissue with immunostimulatory polynucleotide complexed to the carrier, polynucleotides in which immunostimulatory elements are reduced, absent, or masked complexed with carrier typically induce an immune suppressive response, and/or increase expression of IDO in cells while reducing systemic levels of IFNγ and/or activation of natural killer cells. In some embodiments, polynucleotides in which immunostimulatory elements are reduced, absent, or masked complexed with a carrier induce IDO expression with only a local increase in IFNγ at the target site. Because polynucleotides in which immunostimulatory elements are reduced, absent, or masked complexed with a carrier increase IDO expression in cells without a global or systemic increase in IFNγ, in some embodiments it may be necessary to measure global or systemic levels of IFNγ, for example by measuring IFNγ in plasma or serum of the subject.

### IV. Gene Therapy

The compositions including polynucleotides in which immunostimulatory elements are reduced, absent, or masked complexed to a carrier described herein are useful for delivering polynucleotides to cells in a subject with reduced toxicity and potentially adverse reactions. The compositions can be administered to a cell or a subject, as is generally known in the art for gene therapy applications. In gene therapy applications, the compositions are introduced into cells in order to transfect the cell. “Gene therapy” includes both conventional gene therapy where a lasting effect is achieved by a single treatment, and the administration of gene therapeutic agents, which involves the one time or repeated administration of a therapeutically effective polynucleotide. Polynucleotides useful in gene therapy application include, but are not limited to, vectors which may include appropriate expression control elements (e.g., promoter, enhancer, sequences, transcription terminators, polyadenylation sites, etc.), and a selectable marker; and inhibitory nucleic acids such as antisense oligonucleotides, siRNA, miRNA, or RNAi.

For gene therapy application, systemic administration is typically carried out parenterally. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

### EXAMPLES

### Example 1

### pDNA/PEI Nanoparticles Induce Rapid IDO Expression in Mice

### Materials and Methods

Mice

All mice were bred in a specific pathogen-free facility and the local (GHSU) Institutional Animal Care and Use Committee approved all procedures involving mice. A1, OT-1, and OT-2 TCR transgenic mice used in suppression assays were described previously (Baban, et al., J Immunol, 187: (2011)).

DNA/PEI Treatment

Bacteria plasmid (pEGFPN1, invitrogen) DNA (pDNA) was prepared using an endotoxin-free Kit (QIAGEN), CpGfree pDNA (pGIANT) and poly dA:dT (pAT) were purchased from Invivogen, invivo-JetPEI™ was purchased from Polyplus through VWR. DNA-PEI nanoparticles were prepared according to manufacturer's instructions. Mice were injected with 30 μg of DNA complexed with PEI at N:P ratio 10:1 via tail veins.

Immunohistochemistry

Tissue sections (5 μm) were prepared from formalin-fixed paraffinembedded tissues, and subjected to antigen retrieval (Dako Target Retrieval solution, Cat. No. S 1699). Sections were pre-treated with blocking reagents, and incubated with polyclonal rabbit anti-murine IDO antibody (Biosource International, Hopkinton, Mass.) as described (Baban, et al., J Reprod Immunol 61:67-77 (2004)). Stained sections were treated with biotinylated goat anti-rabbit Ig (HK336-9R, BioGenex) and then peroxidase-conjugated streptavidin (HK330-9K, BioGenex). IDO-expressing cells were visualized using 3-amino-9-ethylcarbazole chromogen (>30s<5 min; HK121-5K Liquid AEC, BioGenex), and counterstained with hematoxylin (7221, Richard-Allan Scientific, Kalamazoo, Mich.) and mounted in Faramount (53025, DAKO). IDO antibody pre-incubated with neutralizing peptide was used as a specificity control. For immunofluorescence staining, fresh frozen sections (7 μm) were prepared from O.C.T compound embedded tissues. Sections were fixed, and pre-treated with 1% non-fat milk in PBS (blocking buffer), incubated with primary (1.5 hrs) and secondary (1 hr) antibodies, and mounted (Profade Gold anti-fade mounting media with DAPI, Invitrogen). CD11c antibody was purchased from Biolegend (San Diego, Calif.). Cy3 conjugated goat-anti-rabbit antibody, Dylight 488 conjugated goat-anti-armenian hamster antibody and AMCA conjugated donkey-anti-rat antibody was from Jackson Immuno.

### Results

Few cells in peripheral lymphoid tissues express IDO during homeostasis. While conducting DNA transduction experiments using a commercial source of PEI (invivo-JetPEI™) as a gene transfer vehicle in mice abnormally high levels of IDO staining were detected in mucosal tissues (lungs, GI-tract; not shown) and peripheral lymphoid tissues (spleen, LNs) 24 hours after systemic delivery (i/v) of DNA/PEI nanoparticles containing bacterial plasmid DNA (pDNA). Dispersed clusters of IDO-expressing cells were located exclusively in peri-follicular regions of spleen and peripheral LNs, and most stained cells exhibited plasmacytoid morphologies. The pattern of IDO expression induced by pDNA/PEI nanoparticles closely resembled staining patterns observed in mice treated with the IDO inducers CTLA4-Ig and CpG oligonucleotides (Baban, et al., Int Immunol, 17:909-919, Mellor, et al., J Immunol, 175:5601-5605 (2005), Johnson, et al., Natl Acad Sci USA, 107:10644-10648 (2010)). These reagents ligate B7 and TLR9 respectively, to induce IDO in a small subset of DCs co-expressing the B cell marker CD19. Similarly, IDO-expressing cells in spleen and lymph nodes of pDNA/PEI-treated mice co-expressed the DC marker CD11c, though some IDO-expressing cells did not co-express CD11c, particularly in spleen. Thus pDNA/PEI nanoparticle treatment stimulated rapid IDO expression by DCs and other cell types located in peri-follicular regions of lymphoid tissues.

### Example 2

### Therapy with pDNA/PEI Nanoparticles Suppresses T Cell Responses Elicited In Vivo

### Materials and Methods

In Vivo Suppression Assays

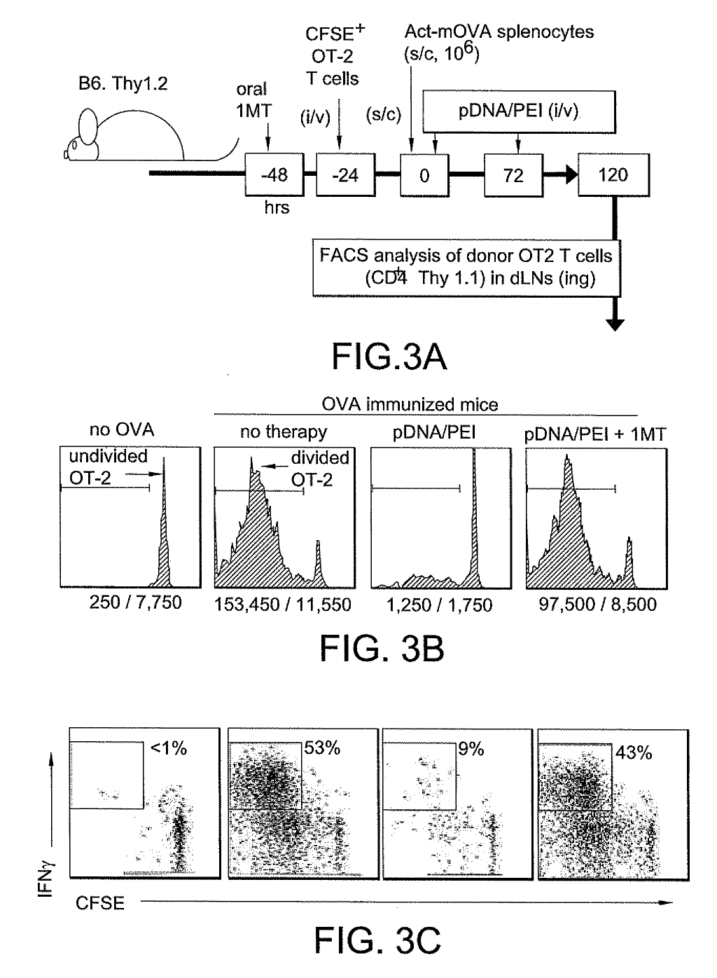

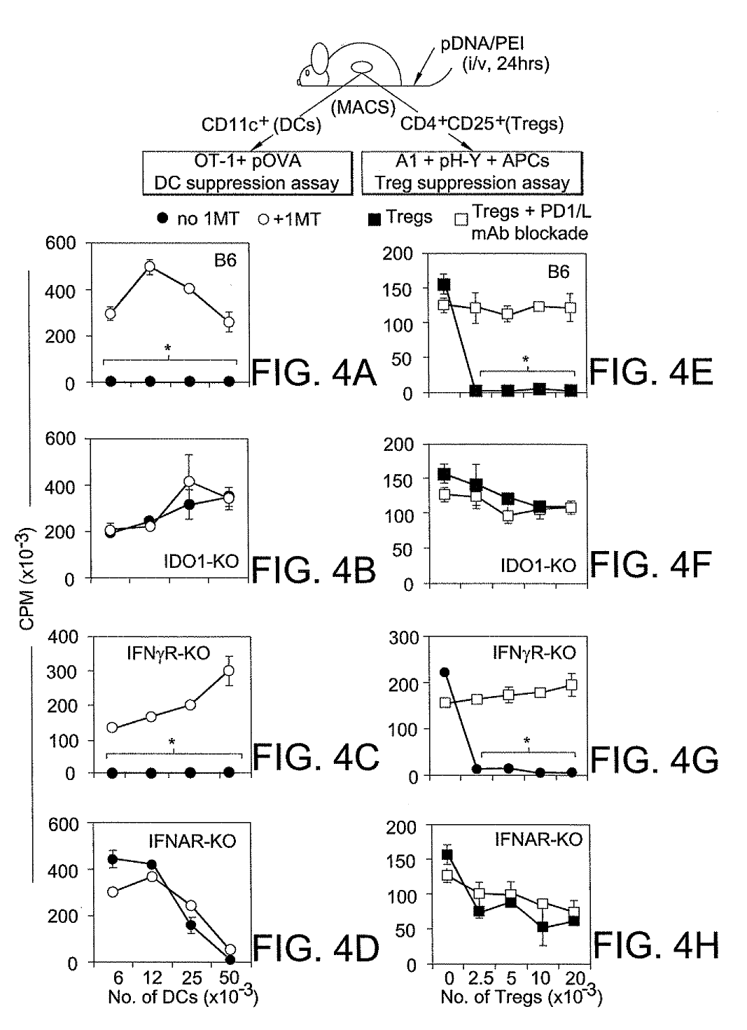

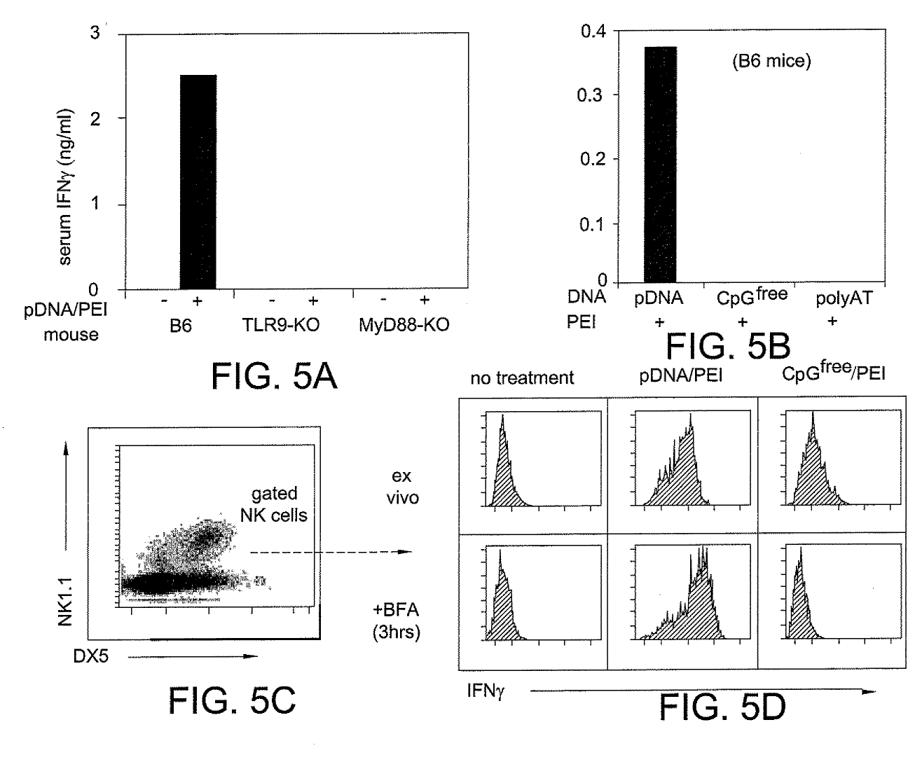

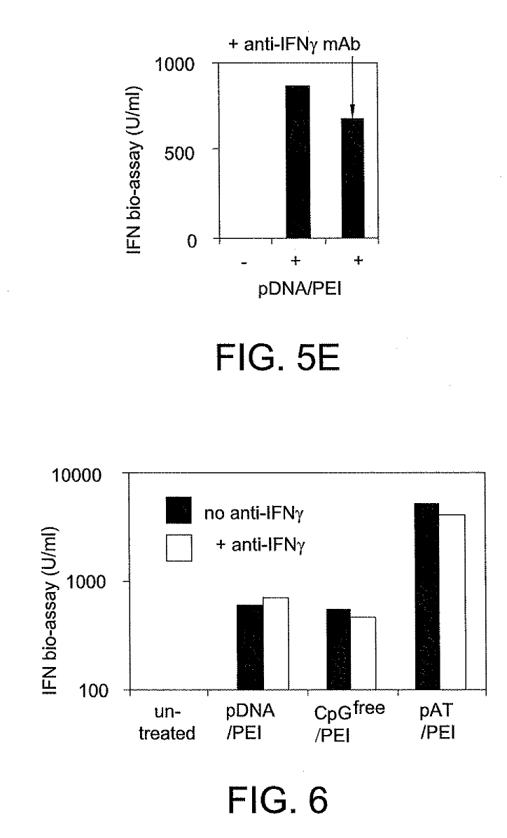

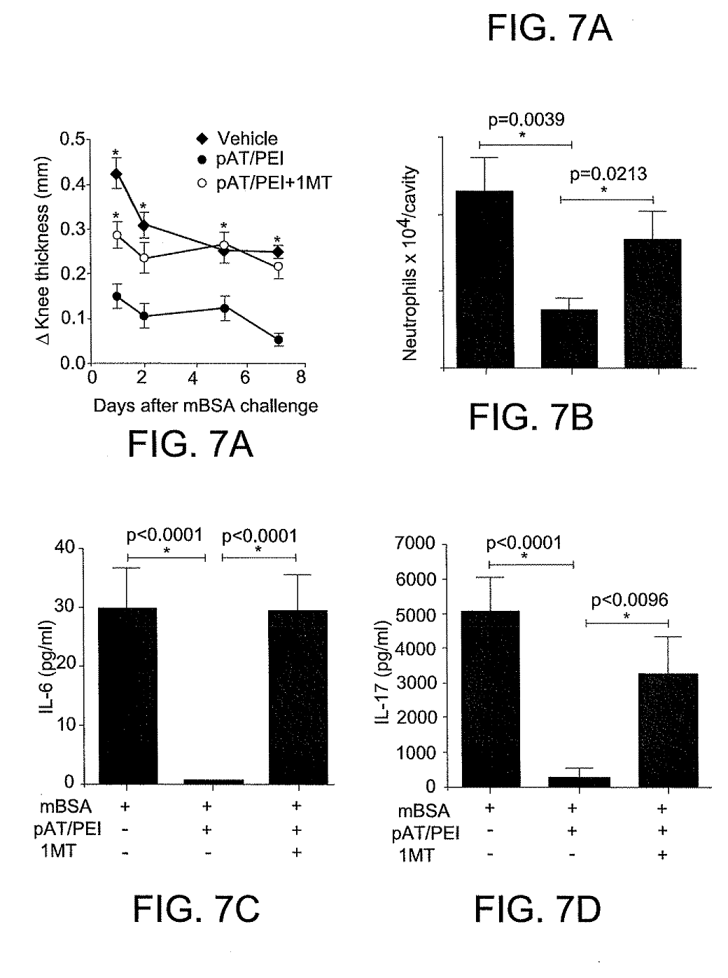

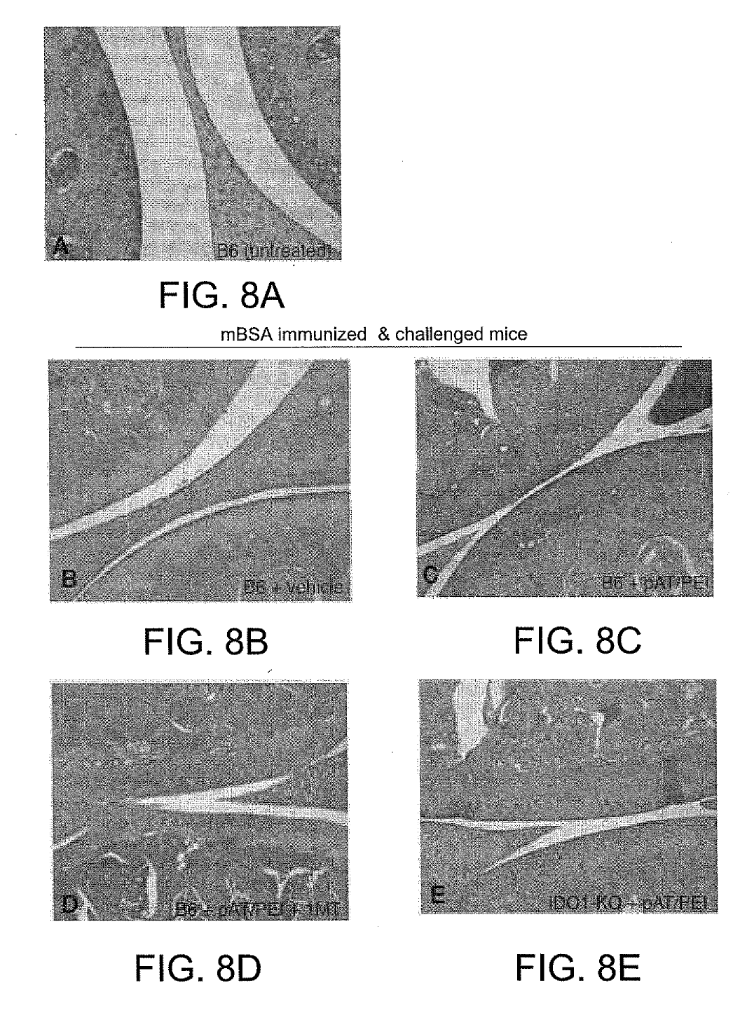

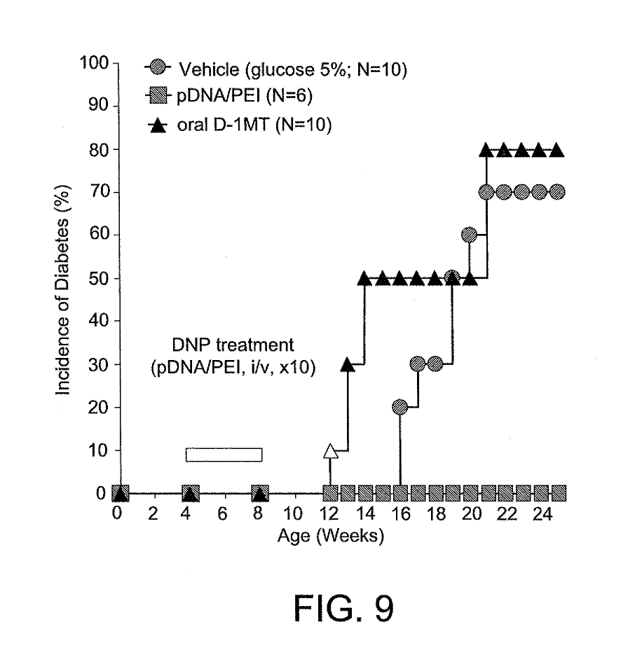

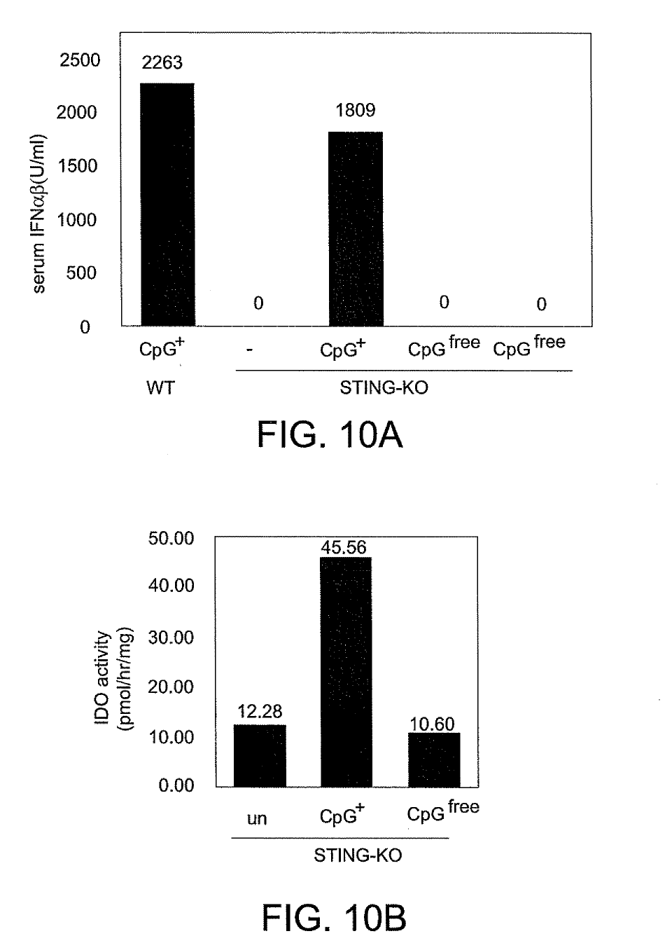

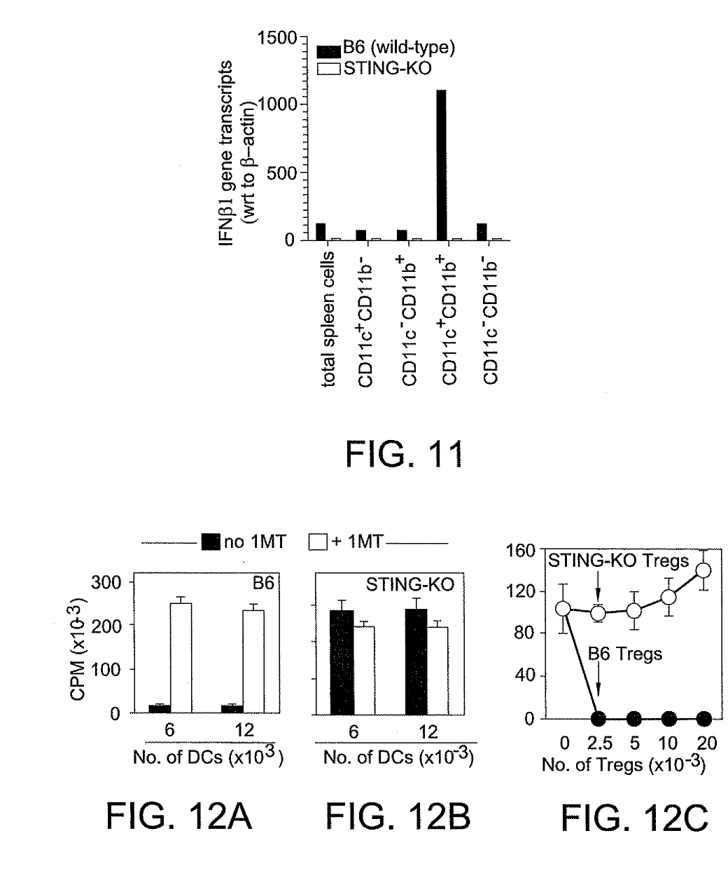

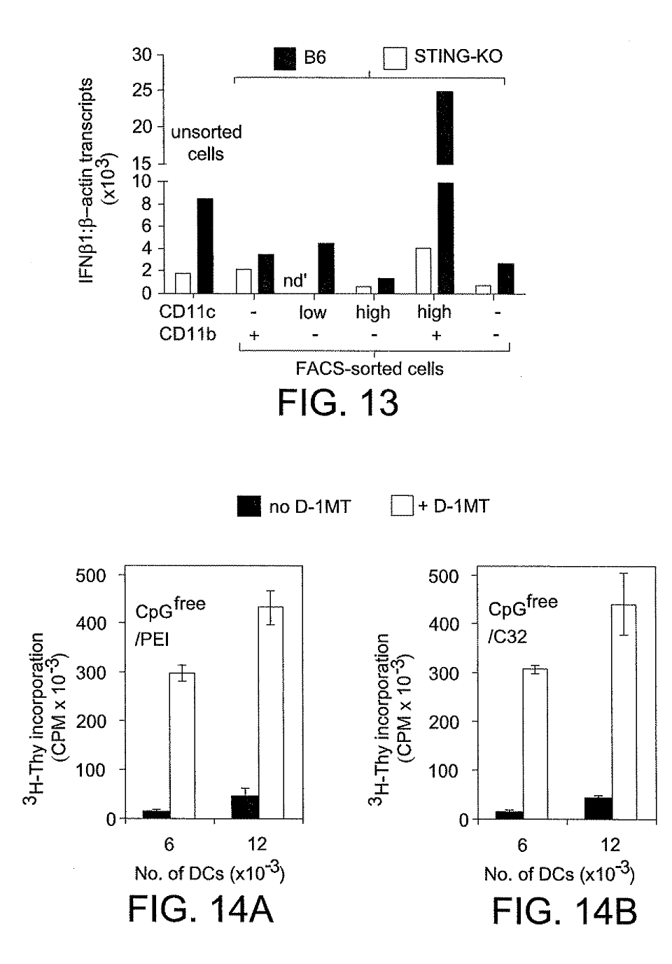

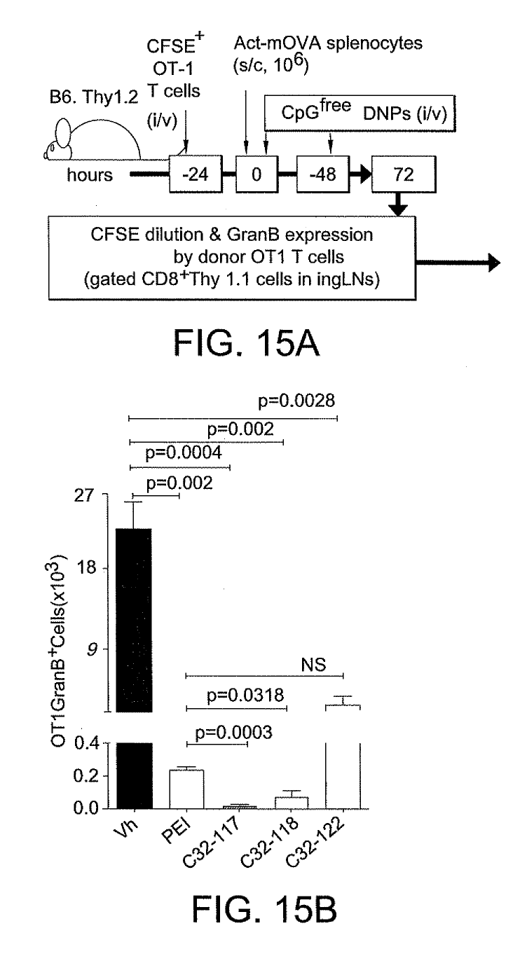

CFSE-labeled OVA-specific T cells from OT-1 or OT-2 donor mice were injected (i/v) into recipient B6 mice at least 1 day before immunization. CFSE labeling was performed by incubating MACS-enriched splenic CD8+ (OT-1) or CD4+ (OT-2) T cells at ˜10×10⁶ cells/ml in PBS with 2 μM CFSE at 37° C. for 15 mins Cells were washed in PBS and injected into recipients (i/v). Mice were then immunized with 106 erythrocyte-free spleen cells from Act-mOVA transgenic mice (s/c), and treated with pDNA/PEI nanoparticles and oral 1MT as indicated in FIG. 3.

Statistical Analysis

All statistical analysis were performed with unpaired Student's t test using Graphpad Prism.

### Results

When induced to express IDO DCs acquired potent regulatory phenotypes that inhibited proinflammatory cytokine expression, suppressed effector T cell responses and activated Tregs (Baban, et al., J Immunol, 183:2475-2483 (2009), Baban, et al., J Immunol, 187: (2011)). To test if pDNA/PEI nanoparticle treatment inhibited T cell responses ovalbumin (OVA)-specific CD4 T cell (OT-2, Thy1.1) responses to OVA immunization were monitored. CFSE-stained OT-2 T cells were adoptively transferred into B6 (Thy1.2) recipients and 1 day later mice were immunized with OVA+ splenocytes from Act-mOVA transgenic mice (FIG. 3A). As expected, five days after OVA immunization OT-2 T cells had proliferated, and many had differentiated into effector TH1 cells expressing IFNγ in inflamed inguinal draining LNs (dLNs) at sites of OVA immunization (FIGS. 3B and 3C, second panels from left). In contrast, clonal expansion and differentiation of OT-2 T cells was suppressed significantly in dLNs from OVAimmunized mice treated with pDNA/PEI nanoparticles (FIGS. 3B, 3C, third panels from left). Oral dosing with the IDO-specific inhibitor 1-methyl-[D]-tryptophan (1MT) starting 2 days before OVA immunization abrogated the regulatory effects of pDNA/PEI treatment (FIGS. 3B, 3C, right panels). Comparable outcomes were obtained in experiments using OVA-specific OT-1 (CD8) T cells instead of OT-2 T cells (not shown). Collectively, these findings revealed that pDNA/PEI treatment blocked in vivo T cell responses to exogenous immunizing antigen, and that IDO activity was essential for T cell regulatory effects to manifest in vivo after pDNA/PEI treatment.

### Example 3

### pDNA/PEI Treatment Induces DCs and Tregs to Acquire Regulatory Phenotypes Via IDO

### Materials and Methods

### Analytical Flow Cytometry

Cells were stained with the following antibodies; anti-CD4 (clone RM4-5), anti-Thy1.1 (clone OX-7), anti-NK1.1 (clone PK136), anti-CD49b (clone DX5), anti-IFNγ (clone XMG1.2) from Pharmingen-BD-Biosciences (San Jose, Calif.), and analyzed using a LSR2 flow cytometer (Becton-Dickinson). CFSE was purchased from Invitrogen. For detecting intracellular IFNγ, cells were surface stained with anti-NK1.1 and anti-DX5, fixed with cytofix/cytoperm, washed with Perm/Wash solution (BD Bioscience) and stained with anti-IFNγ. Some cells were incubated in RPMI with 1 μM of brefeldin (BD Bioscience) for 3 hrs. with no further stimulations to accumulate cytokine before staining. For detecting IFNγ in OT-2 T cells, dLN cells were stimulated with PMA/ionomycin for 2 hrs. with brefeldin. Cells were then surface stained with anti-CD4, anti-Thy1.1 followed by intracellular staining for IFNγ.

DC and Treg Suppression Assay

MACS-enriched splenic DCs (CD11c+) and Tregs (CD4+CD25+) were isolated according to manufacturer's instructions, except that cells were incubated with beads at RT (Magnetic Cell Separation Technology (MACS)—Miltenyi Biotec Inc., Auburn, Calif.). T cell stimulatory activity of DCs was assessed by culturing graded numbers of DCs (X-Y) with responder (MACS-enriched) CD8+ T cells from OT-1 (+SIINFEKL peptide) (SEQ ID NO:1) TCR-Tg mice+/−1MT as described (11). Suppressor activity of splenic Tregs was assessed by culturing graded numbers of Tregs with responder A1 (H-Y-specific) T cells, APCs (female CBA) and male (H-Y) peptide; a cocktail of anti-PD-1, anti-PD-L1, and anti-PD-L2 mAbs was added to some cultures to block PD-1-PD-L interactions as described (Baban, et al., J Immunol, 183:2475-2483 (2009), Sharma, et al., J Clin Invest 117:2570-2582 (2007)).

### Results

To evaluate if pDNA/PEI treatment induced DCs to acquire T cell regulatory phenotypes mice were treated with pDNA/PEI nanoparticles and 24 hrs. later graded numbers of MACS-enriched splenic CD11c+ DCs from pDNA/PEI-treated mice (FIG. 4, left) were cultured with OVA-specific OT-1 T cells and cognate peptide (pOVA, SIINFEKL (SEQ ID NO:1)) as described (Mellor, et al., J Immunol, 175:5601-5605 (2005)). As expected, DCs from untreated B6 mice stimulated robust T cell proliferation, and 1MT had no significant effect on T cell responses elicited ex vivo (not shown). In striking contrast, OT-1 T cells did not proliferate when cultured with DCs from pDNA/PEI-treated mice. Adding 1MT restored robust T cell responses, revealing that DCs actively suppressed T cell proliferation via IDO (FIG. 4A). Thus splenic DCs acquired potent T cell regulatory phenotypes due to induction of IDO after pDNA/PEI treatment. Consistent with this result, DCs from pDNA/PEI-treated mice lacking intact IDO1 genes (IDO1-KO mice) stimulated robust OT-1 T cell proliferation and adding 1MT did not further enhance OT-1 proliferation (FIG. 4B).

DCs expressing IDO activated and sustained potent regulatory phenotypes of Foxp3-lineage Tregs in tumor draining LNs (Sharma, et al., J Clin Invest, 117:2570-2582 (2007)). Similarly, IDO-expressing splenic DCs in mice treated with TLR9 ligands (CpGs) induced functionally quiescent (resting) Tregs to acquire potent regulatory phenotypes (Baban, et al, J Immunol 183:2475-2483 (2009), Baban, et al., J Immunol, 187: (2011)). To test if pDNA/PEI treatment activated Tregs via IDO, graded numbers of MACS-enriched splenic CD4+CD25+ cells (enriched for Foxp3+ Tregs) from pDNA/PEI-treated B6 mice were added to cultures containing responder male (H-Y) antigen-specific CD4+ T cells from A1 TCR-Tg mice, cognate H-Y peptide, and antigen presenting cells (APCs) from CBA female mice (FIG. 4, right). As reported previously (Baban, et al, J Immunol 183:2475-2483 (2009), Sharma, et al., J Clin Invest, 117:2570-2582 (2007)), these assays contain no Treg mitogens, and Tregs (B6) are genetically mismatched with responder T cells and APCs (CBA).

Hence, Tregs cannot activate ex vivo, and suppression must be generated in vivo. As expected, ‘resting’ Tregs from untreated mice had no effect on proliferation of A1 T cells (not shown). In contrast, Tregs from B6 mice treated for 24 hrs. with pDNA/PEI nanoparticles suppressed A1 T cell proliferation completely (FIG. 4E, closed symbols). Robust A1 T cell proliferation was restored in the presence of a cocktail of mAbs that block interactions between programmed death ligand-1 (PD-1), and its ligands, PD-L1 and PD-L2 (FIG. 4E, open symbols). These outcomes revealed that pDNA/PEI treatment induced Tregs to acquire potent regulatory phenotypes rapidly via IDO since PD-1/PD-L-dependent suppression is a hallmark feature of IDO-activated Tregs (Baban, et al, J Immunol 183:2475-2483 (2009), Sharma, et al., J Clin Invest, 117:2570-2582 (2007)). Consistent with these results, Tregs from pDNA/PEI-treated mice lacking intact IDO1 genes did not suppress A1 T cell proliferation ex vivo, and adding PD-1/L blocking mAbs did not further enhance A1 proliferation (FIG. 4F). Thus, pDNA/PEI treatment induced DCs and Tregs to acquire potent regulatory phenotypes rapidly via IDO.

### Example 4

### DNA/PEI Nanoparticles Induce IDO Via IFN Type I Dependent, IFNγ Independent Signaling

### Materials and Methods

Kynurenine and IDO Enzyme Activity Detection in Tissues

Snap frozen tissues were homogenized in PBS at 100 mg/ml (spleen) and 50 mg/ml (lymph nodes). For tissue kynurenine concentration analysis, homogenates were acidified by adding 1/10 volume of 150 mM sodium acetate, pH4.0, and then de-proteinated with 25% volume of 30% trichloride-acetic acid. Kynurenine and tryptophan were measured using HPLC with a C18 reverse phase column as described. Assays to detect IDO enzyme activity in cell-free tissue homogenates were performed as described (Hoshi, et al., J Immunol, 185:3305-3312 (2010)). Briefly, tissue homogenates were mixed with assay solution containing, and reaction mixtures were incubated at 370 C for 2 hrs. Kynurenine concentrations were measured using HPLC before and after incubation. IDO activity was calculated as pmol of kynurenine generated per hour of reaction time per mg tissue. 1-methyl-[D]-tryptophan (1MT). 1MT (Newlink Genetics Inc.) was prepared as a 20 mM stock solution in 0.1M NaOH, adjusted to pH 7.4, and stored at −20° C. protected from light. For in vitro use, 1MT was added at a final concentration of 100 μM. For in vivo treatment mice were provided with 1MT (2 mg/ml) in drinking water with sweetener (Nutrasweet) to enhance palatability as described (Hou, et al., Cancer Res, 67:792-801 (2007)).

### Results

IFN types I (IFNαβ) and II (IFNγ) are potent IDO inducers, though requirements for IFN signaling vary in different cell types. To evaluate IFN signaling requirements to induce IDO after pDNA/PEI treatment the effects of ablating IFN type I (IFNAR) and type II (IFN′R) receptor genes on induced regulatory phenotypes in DCs and Tregs were assessed. DCs (FIG. 4C) and Tregs (FIG. 4G) from IFNγR-KO and B6 (wt) mice mediated T cell suppression after pDNA/PEI treatment with comparable potencies. However, DCs (FIG. 4D) and Tregs (FIG. 4H) from pDNA/PEI-treated IFNAR-KO mice did not mediate T cell suppression. Thus IDO induction in DCs was dependent on signaling via IFN type I receptors while IFNγ receptor signaling was not essential for this response.

To further evaluate IFN signaling requirements to induce IDO production of kynurenine (Kyn)—a tryptophan catabolite released by cells expressing IDO—was measured in tissue lysates from pDNA/PEI-treated mice. pDNA/PEI treatment elevated Kyn levels in spleen and LNs ˜10-fold and ˜4-fold, respectively over basal levels in untreated mice (Table 1). In addition, IDO enzyme activity—measured as Kyn produced by cell-free tissue extracts in enzyme substrate cocktail (Hoshi, et al., J Immunol 185:3305-3312 (2010))—increased significantly in spleen (˜5-fold) from pDNA/PEI-treated mice (Table 1).

Collectively, these data revealed that IDO induction leading to immune regulatory outcomes was a rapid and potent response to pDNA/PEI nanoparticle treatment.

### Example 5

### pDNA/PEI Nanoparticles Induce Systemic IFNγ Production and Activate NK Cells Via TLR9

Consistent with a recent report (Rodrigo-Garzon, et al., Cancer Gene Ther, 17:20-27 (2010)), large increases in serum IFNγ were detected 24 hrs. after B6 mice were treated with pDNA/PEI nanoparticles (FIG. 5A). However these rapid responses did not occur in mice with defective TLR9 and MyD88 genes after pDNA/PEI treatment (FIG. 5A). It is believed that un-methylated CpG motifs in bacterial pDNA are responsible for elevating serum IFNγ via the TLR9/MyD88-dependent signaling pathway. DNA nanoparticles lacking un-methylated CpG motifs—due to incorporation of CpGfree pDNA or synthetic poly dA:dT (pAT) oligomers into DNA/PEI nanoparticles rather than bacterial pDNA—did not stimulate rapid increases in serum IFNγ in B6 mice (FIG. 5B).

To identify cells induced to express IFNγ intracellular IFNγ expression in spleen by flow cytometric analyses 3 hrs. after mice were treated with pDNA/PEI nanoparticles was assessed. Induced intracellular IFNγ was detected exclusively in cells expressing the NK cell markers NK1.1, DX5 after DNA/PEI treatment (FIG. 5C, 5D, and data not shown). Gated NK cells (FIG. 5C) uniformly expressed IFNγ when analyzed to detect intracellular IFNγ directly (ex vivo), or after culture for 3 hrs. in the presence of brefeldin (BFA) to block protein secretion (FIG. 5D, center histograms).

In contrast, splenic NK cells from mice treated with CpGfree pDNA/PEI nanoparticles expressed little IFNγ relative to gated NK cells from untreated mice (FIG. 5D, right and left histograms, respectively). Thus pDNA/PEI nanoparticles stimulated rapid activation of splenic NK cells leading to IFNγ release, and this innate immune response was dependent on the presence of TLR9 ligands in pDNA/PEI nanoparticles.

Thus IFNγ production was dependent on TLR9-mediated DNA sensing. As IFNα is a potent IDO inducer in some cell types—including IDO-competent CD19+ DCs (Mellor, et al., J Immunol, 175:5601-5605 (2005))—serum was tested for IFN type I activity using bioassays (Lcell/VSV infection interference) with & without anti-IFNγ mAbs. Consistent with IFNγ ELISA assays elevated IFN activity was detected in serum from pDNA/PEI-treated mice; however only ˜20% of IFN activity was due to IFNγ (FIG. 5E), Thus IDO induction was not TLR9-dependent, and DNA/PEI nanoparticles lacking CpG motifs induced IFN type I but not IFNγ.

DNA/PEI nanoparticles activate DC2.4 cells. DC2.4 cells were incubated with DNA/PEI nanoparticles for 18 hrs. and IFN activity in media was assessed. Untreated DC2.4 cells expressed no IFN activity, while IFN activity was present in media from DC2.4 cells treated with DNA/PEI nanoparticles containing pDNA, CpGfree pDNA or polyAT DNA (FIG. 6). FITC-labeled nanoparticles containing polyAT DNA were ingested faster than other DNA/PEI nanoparticles (not shown) providing a potential reason why ˜10-fold higher IFN activity was induced by polyAT/PEI nanoparticles. Thus DNA/PEI nanoparticles induced IFN type I via a TLR9-independent DNA sensor in DC2.4 cells. In a previous study, TLR9 ligands (CpG ODNs) induced bone-marrow derived DCs (BMDCs) and DC2.4 cells to express pro-inflammatory cytokines, while vertebrate DNA complexed with cationic lipids did not (Yoshinaga, et al., Immunology, 120:295-302 (2007)). Thus DNA/PEI nanoparticles may be more effective in activating DCs, though co-induced IDO may mediate dominant regulation in vivo.

### Example 6

### DNA/PEI Nanoparticles Lacking TLR9 Ligands Induce Functional IDO Expression

Since IFNγ is essential for IDO-dependent regulatory responses to DNA/PEI nanoparticles (FIG. 4 & Table 1), DNA/PEI nanoparticles lacking TLR9 ligands were tested to determine if they could still induce IDO. Treatment with DNA/PEI nanoparticles containing CpGfree pDNA or pAT stimulated significant increases in tissue Kyn and IDO enzyme activity in spleen (Table 1) and LNs (data not shown) of B6 mice. Moreover, pDNA/PEI nanoparticle treatment suppressed OT-1 T cell proliferation and differentiation via IDO following OVA immunization to comparable extents in B6 mice (as shown in FIG. 3) and in TLR-9-deficient mice (data not shown). Collectively, these data revealed that systemic IFNγ release was caused by TLR9 ligands in pDNA/PEI nanoparticles, and that systemic IFNγ production was not essential to induce IDO and consequent potent suppression of T cell responses elicited in vivo.

TABLE 1
Kynurenine and IDO activity lymphoid tissues

DNA/PEI nanoparticles

| | pDNA | | | | Kynurenine | IDO activity |
|---|---|---|---|---|---|---|
| Mouse | CpG | CpG-free | pAT | Tissue | (pmol/g)^{a} | (pmol/mg/hr)^{a} |
| B6 | − | − | − | spleen | 0.7 ± 0.3 | 19.1 ± 2.7 |
| B6 | + | − | − | spleen | [symbol] | [symbol] |
| B6 | − | − | − | LNs | 2.6 ± 1.2 | nt |
| B6 | + | − | − | LNs | [symbol] | nt |
| B6 | − | + | − | spleen | [symbol] | [symbol] |
| B6 | − | − | + | spleen | [symbol] | [symbol] |
| TLR9-KO | + | − | − | spleen | 12.7 | 53.2 |
| TLR9-KO | − | + | − | spleen | 12.1 | 57.0 |

Notes: nt, not tested
^{a}mean ± 1sd: p < 0.002-0.0001^{b}, p < 0.013^{c} (treated vs untreated)
results in bold highlight IDO activity significantly above basal levels in untreated mice

### Example 7

### DNA/PEI Nanoparticles Regulate Autoimmune Inflammatory Disease Progression

### Materials and Methods

mBSA-Induced Arthritis Model

B6 mice were sensitized with methylated BSA (mBSA, s/c, 500 mg in CFA (day 0). Booster injections of mBSA in IFA were given on days 7 and 14, and arthritis was induced on day 21 by intra-articular injection of mBSA (10 μg in PBS, challenge).

Arthritis severity was evaluated by measuring joint swelling, neutrophil infiltration, and histological analysis as described (Lemos, et al., Proc Natl Acad Sci USA, 106:5954-5959 (2009)). In brief, joint swelling was assessed using a vernier caliper, and was expressed as the increase in diameter (mm) relative to non-inflamed joints in each mouse at experimental starting points. Neutrophils were counted after harvesting cells from articular cavities 24 hours after mBSA challenge. Results were presented as neutrophil numbers per cavity (mean±SEM). Arthritis was analyzed histologically 28 days after initial immunization (experimental endpoints). Knee joints were dissected and fixed in 10% buffered formalin for 3 days. Fixed tissues were decalcified for 3 days in Decal Stat (Decal Chemical Corporation, New York, USA), dehydrated, and embedded in paraffin. Sagittal sections (5 um) of the knee joint were stained with Safranin-O and counterstained with fast green/iron hematoxylin. Sections were examined by two independent observers and graded blindly using a semi-quantitative score from 0 to 3 (0, no; 1, mild; 2, moderate; 3, severe alterations) for the extent of (a) synovial lining layer hyperplasia and (b) infiltration of leukocytes into synovial membrane/joint space (as measures of joint inflammation); and (c) pannus formation and necrosis/erosion of cartilage (as a measure of joint destruction). The final arthritis score was evaluated for each mouse by calculating the sum of the values for inflammation and destruction (maximal evaluation grade=12). As additional measures of local inflammation popliteal and inguinal LN cells draining inflamed joints were harvested, and cultured (106 cells/well) in the presence or absence of mBSA (100 μg/ml) for 36 hrs. Culture supernatants were harvested, and IL-17 and IL-6 concentrations were measured using a multiplex bead system (Luminex™) according to the manufacturer's instructions.

### Results

A model of antigen-induced rheumatoid arthritis was used to test the effect of DNA/PEI on autoimmunity (Lemos, et al., Proc Natl Acad Sci USA, 106:5954-5959 2009)). B6 mice were immunized with methylated BSA (mBSA/CFA, day 0), then boosted twice with mBSA/IFA (days 7, 14), and local joint arthritis was induced by intra-articular injection of mBSA (challenge) 21 days after initial immunization Immunized mice were treated five times with DNA/PEI nanoparticles (days 20, 21, 22, 24, 26) containing polyAT (pAT) dsDNA to avoid activating NK cells and stimulating systemic IFNγ release, and thereby minimize the risk of inciting toxic effects due to DNA/PEI nanoparticle treatment. Knee joint swelling in mBSA-sensitized mice was reduced significantly in mice that received pAT/PEI treatment (FIG. 7A). The ameliorative effects of pAT/PEI therapy on local joint inflammation were reversed in mice provided with oral 1MT starting two days before initial mBSA immunization until experimental endpoints 7 days after intra-articular mBSA challenge. Similarly, therapeutic effects of pAT/PEI treatment were observed for other measures of arthritis severity, including neutrophil infiltration into joints one day after mBSA challenge (FIG. 7B), and levels of the pro-inflammatory cytokines IL-6 (FIG. 7C) and IL-17 (FIG. 7D) produced ex vivo by cells from inflamed inguinal and popliteal LNs draining sites of joint inflammation. In each case, pAT/PEI treatment reduced disease parameters significantly, and oral 1MT fully or partially abrogated the therapeutic effects of pAT/PEI treatment. Consistent with these outcomes, sulfated cartilage proteoglycan loss—a key indicator of joint destruction—was reduced significantly when analyzed 7 days after local mBSA challenge, and oral 1MT treatment eliminated the therapeutic effect of pAT/PEI nanoparticles by this measure (FIGS. 7E-G).

Thus, treatment with DNA/PEI nanoparticles lacking immunostimulatory DNA elements that trigger systemic release of IFNγ attenuated innate and adaptive immunity that drives joint destruction.

In summary, it has been discovered that that DNA/PEI nanoparticles elicited potent regulatory responses in mice by inducing IDO. As a consequence dendritic cells (DCs) and Tregs acquired regulatory phenotypes that suppressed innate and adaptive T cell responses to defined exogenous antigens and autoantigens. Removal of immunostimulatory (CpG) motifs in bacterial plasmid DNA abrogated systemic IFNγ production by activated NK cells without compromising IDO-mediated immune regulatory responses induced by DNA/PEI nanoparticles indicating that TLR9 ligands in DNA/PEI nanoparticles are key factors that drive pro-inflammatory responses. Furthermore, treatment with DNA/PEI nanoparticles lacking TLR9 ligands is effective in attenuating antigen-induced autoimmune arthritis via IDO. These results show that regulatory responses via IDO are key components of rapid inflammatory responses elicited by DNA/PEI nanoparticles, and immunostimulatory CpG motifs in DNA/PEI nanoparticles are essential to elicit systemic IFNγ production by NK cells indicating that the use of bacterial DNA in gene transfer procedures biases physiologic responses towards pro-inflammatory outcomes with potential toxic effects.

### Example 8

### DNA/PEI Nanoparticles Prevents Type I Diabetes

### Materials and Methods

Rat insulin promoter-ovalbumin (RIP-OVA) transgenic mice were bred with IDO-sufficient (WT) and IDO-deficient (IDO1-KO) backgrounds. All mice were injected with OVA-specific CD8 T cells (from OT-1 donor mice), and then immunized with OVA vaccine with or without 5 injections of pAT/PEI (every other day) starting 2 days before OVA immunization. Type 1 Diabetes (T1D) onset was monitored for over 1 month (>30 days).

### Results

Table 2 shows the time of type I diabetes onset after OVA vaccination with or without pAT/PEI treatment in RIP-OVA mice with IDO-sufficient (WT) or IDO-deficient (IDO1-KO) backgrounds; mice were pre-injected with OVA-specific CD8+ T cells (2×10⁴ OT-1 donor T cells)

TABLE 2
Results of poly AT/PEI treatment in a type I diabetes model
in mice.

| IDO1 | DNPs | T1D onset |
|---|---|---|
| (RIP.OVA) | pAT/PEI | (day) |
| WT | − | 7, 7, 7, 7 |
| WT | + | >30 (x2) |
| KO | + | 6, 7 |

Non-obese diabetic female (NODf) mice were given 8 injections of pDNA/PEI (i/v, 15 μg pDNA+3 μl invivoJet-PEI™ per injection) over a period of 4 weeks (every 2-3 days) from age 8-12 weeks and then monitored for T1D onset. pDNA/PEI treatment prevents type I diabetes (T1D) progression (FIG. 10 (-▪-)). IDO slows T1D progression in a subset of NODf mice (FIG. 10 (-▴-)), as IDO inhibitor accelerated disease onset in at least 50% of NOD female mice.

### Example 9

### DNPs Associate Rapidly with Discrete Subsets of Mfs and DCs Located in the Marginal Zone (MZ) of Mouse Spleen

### Materials and Methods

B6 mice were treated with DNPs (i/v) containing dye-labeled (rhodamine, red) polyethylenimine (i/v). After 3 hrs., spleen sections were stained (FITC, green) to detect MOMA1⁺ (CD169) MZ macrophages (MFs) and CD11c⁺ dendritic cells (DCs). After 24 hrs., FACS-sorted MZ MOMA1⁺ (F4/80^{neg}) MFs were stained to detect IDO.

DNA Nanoparticle Treatment:

Mice were injected (i.v.) with 400 μl of DNA nanoparticles made with 6 μl 150 mM PEI (PEI from Polyplus, France) and 30 mg pGiant (CpG^{free}) DNA (Invivogen, CA).

Flow Cytometry:

Spleens were injected with 2 ml of RPMI containing 400 U/ml of collagenase IV (Worthington-Biochem, NJ), and incubated for 30 min. at 370 C in 5 mL RPMI containing 400 U/mL collagenase. Red blood cells were lysed using ACK lysing buffer (Lonza, Md.). For cell sorting experiments single-cell suspensions were incubated with anti-CD11c and anti-CD11b mAbs (eBioscience, CA). Cells were sorted on a Mo-Flo (Dako Cytomation) cell sorter into tubes containing RNA protection reagent (Omega Bio-tek). For analysis experiments, single-cell suspensions were incubated with anti-CD11c, anti-CD11b, anti-CD8 (eBioscience, CA) and anti-MOMA-1 (Serotec, NC). Cells were analyzed on a LSR11 flow cytometer (Becton Dickinson). DAPI was added prior to analysis to identify dead cells. Data were analyzed using FACS DIVA (BD Bioscience) or FlowJo (Tree Star, Ashland, Oreg.) software.

### Results

DNPs associated rapidly with rare cells located in marginal zones (MZ) surrounding lymphoid follicles. Cells associated with DNPs express MF (CD169) and DC (CD11c) markers DNPs are ingested rapidly by discrete subsets of splenic MZ cells, implying that the potent and dominant immune regulatory effects of DNPs in mice are mediated by small numbers of innate immune cells specialized to regulate immune responses. Such cells may have evolved to ensure that debris (self antigens) from dying (apoptotic) cells do not incite autoimmunity.

### Example 10

### Rare Subsets of Splenic DCs and Non-DCs Ingest DNPs Rapidly

### Materials and Methods

B6 mice were treated with DNPs containing dye-labeled (YoYo) cargo DNA. After 3 hrs, splenocytes were stained with the phenotypic markers CD11C, CD8α, CD11b and analyzed in a flow cytometer.

### Results

Cargo DNA from DNPs accumulated rapidly in small subsets of DCs (CD11c+) and non-DCs (CD11c^{neg}). Cells associated with cargo DNA after (3 hrs) represent <0.1% of total splenocytes and ˜1% of DCs and MFs. CD8—a marker of activated DCs that cross-present antigens—is a prominent marker of DCs that rapidly ingested cargo DNA from DNPs.

These data support the hypothesis that small populations of cells in spleen actively ingest nanoparticles and process ingested material. Implying that these cells also respond to cargo DNA from ingested DNPs to elicit downstream tolerogenic responses via IFN type I and IDO induction via autocrine and/or paracrine signaling pathways independent of TLR9 (as responses still occur in the absence of TLR9 ligation)

### Example 11

### Cells that Ingest DNA Nanoparticles are Candidate ‘First-Responder’ Cells

### Materials and Methods

B6 mice were treated (i.v.) with nanoparticles containing rhodamine-conjugated PEI (Rh-PEI) and bacterial plasmid DNA lacking TLR9 ligands (CpG^{free}). After 3 hours spleen sections were stained to assess the phenotypes of cells that ingested Rh-PEI.

### Results

Most Rh-labeled cells were clustered in MZ and were largely absent in lymphoid follicles. Rh-PEI staining was associated strongly with MOMA-1 (CD169) expression by metallophilic MZ MΦs, and was also associated with smaller subsets of MZ DCs (CD11c) and other MZ cells expressing the monocyte/MΦ marker CD11b.

To further characterize cells that ingested cargo DNA B6 mice were treated with nanoparticles containing DNA (CpG^{free}) labeled with the fluorescent dye YoYo-1 for 1 or 3 hours, the early period when DNA/PEI nanoparticles induce copious cytokine production by innate immune cells. Flow cytometric analyses revealed that few splenocytes (<0.5% of total) contained cargo DNA (YoYo-1⁺) after 1 or 3 hours. About 50% of YoYo-1⁺ cells expressed high (CD11c^{high}) or low (CD11c^{low}) levels of CD11c characteristic of myeloid DCs (mDCs) or pDCs, respectively. Most YoYo-1⁺ mDCs (>90%) expressed the activation marker CD8α or the monocyte/myeloid marker CD11b but not both markers, indicating that two discrete mDC populations (CD8α⁺ or CD11b⁺) ingested cargo DNA rapidly. YoYo-1+ mDCs did not express CD19 and ˜50% of YoYo-1⁺CD8α⁺ mDCs expressed the regulatory DC marker CD103, and most YoYo-1⁺CD11b⁺ mDCs expressed the macrophage marker F4/80 but not CD 103 (not shown). In contrast, few YoYo-1⁺pDCs and non-DCs (CD11c^{neg}) expressed CD8α, and ˜60% of non-DCs expressed CD 11b and the MO marker F4/80. Very few (<10%) gated CD169+ MZ MΦs contained YoYo-1-labeled cargo DNA. Thus, the strong association between Rh-PEI nanoparticles and MOMA-1+(CD169) MZ MΦs may arise because these cells degrade ingested cargo DNA rapidly (while PEI resists degradation), unlike other MZ cells that ingest nanoparticles but do not degrade cargo DNA as rapidly. Splenic MZ contains cells that actively scavenge and endocytose debris from dying cells including chromatin and mitochondria that contain DNA. DNA nanoparticles mimic particulate cellular debris containing DNA, are ingested rapidly by a range of cell types, and have been used widely as non-viral vectors to facilitate gene transfer. Cells that ingest DNA nanoparticles are candidate ‘first-responder’ cells that produce IFNαβ to induce IDO and create robust regulatory responses that manifest in spleen, peripheral lymph nodes and sites of immune-mediated tissue injury. PEI from nanoparticles strongly associated with metallophilic MOMA-1+ MZ MΦs that process apoptotic cells via pathways that help maintain tolerance to their contents, though nanoparticle cargo DNA associated with smaller cohorts of other MZ cells expressing CD11c and CD11b. These rare, but discrete splenic MZ cell populations are potential IFNαβ producers that elicit downstream regulatory responses via IDO in response to cargo DNA sensing via TLR9-independent pathways. Moreover, these cells may also reside in, or circulate to peripheral lymph nodes since DNA nanoparticles induced IDO enzyme activity and robust regulatory responses to vaccines in peripheral lymph nodes as well as spleen.

### Example 12

### DNA Sensing Via the STING Pathway Mediates Regulatory Responses to DNPs

### Materials and Methods

B6 (WT) and STING-deficient (KO) mice were treated for 24 hrs. with DNPs containing (CpG+) or lacking (CpG^{free}) TLR9 μligands in cargo DNA. Serum IFN type I (IFNαβ) was detected using an infection interference bioassay in the presence of IFN type II (IFNα) neutralizing mAbs. IDO activity was assessed in cell-free tissue homogenates by detecting kynurenine in culture media by HPLC.

### Results

STING ablation eliminated IFNαβ release and IDO induction in response to DNPs not containing TLR9 ligands. DNPs containing TLR9 ligands induced IFNαβ and IDO induction in STING-KO mice (FIGS. 10A and 10B). DNA sensing via STING is essential for IFNαβ release that induces DCs to express IDO and acquire potent regulatory phenotypes that suppress T cell responses and activate regulatory T cells (Tregs)

These findings support the hypothesis that DNPs target discrete cell types in splenic marginal zone that are specialized to promote immunologic tolerance by expressing type I IFNαβ and induced IDO.

### Example 13

### Cargo DNA Sensing Via the STING Pathway is Essential to Induce IFNαβ and IDO

### Materials and Methods

B6 (WT) and STING-deficient (KO) mice were treated for 24 hrs. with DNPs containing (CpG+) or lacking (CpG^{free}) TLR9 ligands in cargo DNA. Serum IFNαβ and IDO levels were determined as described above.

### Results

As expected, serum IFNαβ levels and splenic IDO activity increased significantly in B6 mice treated with DNA nanoparticles, relative to basal levels in untreated mice (Table 3). In contrast, serum IFNαβ and splenic IDO activity remained at basal levels in STING-KO mice treated with DNA nanoparticles (Table 3), indicating that intact cytoplasmic DNA sensing pathways via STING were essential to induce IFNαβ release and consequent IDO up-regulation after cargo DNA was ingested by cells. Consistent with this interpretation, IDO activity also remained at basal levels in mice lacking intact IFN type I receptors (IFNAR-KO mice) after nanoparticle treatment (Table 3).

TABLE 3
Cargo DNA sensing via the STING pathway in DCs
induces INFαβ and IDO

| | PEI/DNA | Serum IFNαβ | IDO activity |
|---|---|---|---|
| Mice (n) | (CpG^{free}) | (U/ml) | (pmol/hr/mg) |
| B6 (9) | − | <100 | 9.6 ± 1.4 |
| B6 (10) | + | 2908 ± 436** | 25.9 ± 2.5** |
| STING-KO (4) | − | <100 | nd |
| STING-KO (3) | + | <100 | 7.6 ± 2.3 |
| IFNAR-KO (1) | − | <100 | nd |
| IFNAR-KO (3) | + | nd | 9.5 ± 5.3 |
| CD11c^{OTR} + DT (1) | − | <100 | 5.1 |
| CD11c^{OTR} + DT (2) | + | 182 ± 134 | 7.3 ± 2.1 |
| CD169^{OTR} + DT (2) | − | <100 | 5.7 ± 0.5 |
| CD169^{OTR} + DT (3) | + | 1952 ± 295* | 26.3 ± 4.4* |

Notes.
*p < 0.0001;
**p < 0.05;
nd, not done

### Example 14

### DNPs Induce Selected DCs to Express IFN Type I

### Materials and Methods

B6 or STING-KO mice were treated with DNPs (i/v, no TLR9 ligands). After 3 hrs spleen cells were stained with CD11c and CD11b (a monocyte marker) mAbs and sorted in a flow cytometer (FACS). Sorted cells were used to prepare RNA for quantitative RT-PCR analysis to detect I IFNβ1 and β-actin gene transcripts. Data shows relative levels of IFNβ1 transcripts normalized to b-actin levels in each sorted cell type.

### Results

DNPs induced IFNβ1 gene expression in a small subset of DCs expressing the monocytic marker CD11b. Most DCs (CD11c⁺CD11b^{neg}) and most monocytic (MF) cells did not express IFNβ1 in DNP-treated mice (FIG. 11). STING gene ablation eliminated IFNβ1 expression in response to DNPs.

These data show that cytoplasmic DNA sensing via the STING pathway is essential for DNPs to induce innate immune cells to express IFNβ1, the obligate upstream inducer of IDO.

Cells that produce IFNβ1 are a rare subset of DCs co-expressing CD11b, suggesting that these DCs are not pDCs or highly phagocytic CD8⁺ DCs that cross present antigens and do not express CD11b; the frequency of DCs that respond to DNPs in spleen is ˜0.03% (˜1% of DCs). DNPs target a highly specialized innate immune cell type located in the marginal zones of lymphoid tissues that mediate dominant T cell regulation via IDO by making IFNβ1 in response to cytoplasmic DNA sensing via the STING pathway.

### Example 15

### STING DNA Sensing is Essential for Regulatory Responses to DNA Nanoparticles

### Materials and Methods

B6 or STING-KO mice were treated with nanoparticles containing CpG^{free} cargo DNA, and 24 hours later graded numbers of MACS enriched splenic DCs were cultured with OVA-specific OT-1 responder T cells and OVA peptide as previously described.

DC and Treg Suppression Assays.

Assays to detect IDO-dependent T cell regulatory phenotypes in splenic DCs were performed as previously described. Briefly, MACS enriched (CD11c⁺) spleen cells were cultured for 72 hours with MACS-enriched CD8α⁺ T cells from OT-1 TCR transgenic mice and OVA257-264 peptide (SIINFEKL (SEQ ID NO:1)), 100 nM). Proliferation was assessed by adding 1 μCi methyl-[³H]-thymidine for the final 6 hours and quantified using a BetaPlate counter (Wallac). 1-methyl-[D]-tryptophan (1MT) was used at a final concentration of 100 μM. Assays to detect regulatory phenotypes in Tregs were performed as previously described. Briefly, MACS-enriched (CD4⁺CD25⁺) splenic Tregs were cultured for 72 hours with MACS-enriched CD4⁺ T cells from A1 (H-2Ek restricted, H-Y-specific) TCR transgenic mice, and CD11c⁺ spleen cells from CBA female mice and cognate H-Y peptide (REEALHQFRSGRKPI (SEQ ID NO:2), 100 nM). Proliferation was assessed as described above.

### Results

As expected, DCs from treated B6 mice did not stimulate OT-1 T cell proliferation, unless the IDO-specific inhibitor 1-methyl-tryptophan (1MT) was added to cultures (FIG. 12A), indicating that DCs acquired robust T cell regulatory phenotypes via IDO in response to DNA nanoparticle treatment. In contrast, DCs from treated STING-KO mice stimulated robust OT-1 T cell proliferation, which was not enhanced by adding 1MT (FIG. 12B), indicating intact STING signaling was required for nanoparticle cargo DNA to elicit innate immune responses that caused DCs to acquire regulatory phenotypes via IDO. Consistent with these findings, splenic Tregs from STING-KO mice treated with DNA nanoparticles did not exhibit T cell regulatory phenotypes ex vivo, while Tregs from control B6 mice exhibited potent regulatory phenotypes such that only 2500 Tregs prevented proliferation of a large excess of responder A1 T cells (Treg:Teffectors=1:20). Thus, cytoplasmic DNA sensing and signaling via STING to induce IDO in DCs that activates Tregs is essential to induce regulatory responses to DNA nanoparticles.

Most (perhaps all) cells can sense cytoplasmic DNA and trigger IFNαβ release via the STING pathway, and such responses are critical to elicit effective host immunity to certain DNA viruses. Excessive DNA sensing via the STING pathway also provoked systemic IFNαβ-mediated autoimmunity in mice lacking Trexl DNA degrading activity, indicating that constitutive DNA degradation by Trexl was essential to prevent chronic STING activation in non-hematopoietic cells that incited systemic IFNαβ-mediated autoimmunity. In contrast, other data show that STING activation in MZ cells that ingested nanoparticle cargo DNA regulated T cell responses to vaccines and suppressed immune-mediated tissue injury. Accordingly, the data herein support the hypothesis that constitutive DNA sensing and STING activation in MZ cells specialized to scavenge dying cells triggers IFNαβ-mediated regulatory responses via IDO that maintain tolerance and suppress autoimmunity to DNA. Consistent with this hypothesis, pharmacologic and genetic ablation of IDO led to increased lupus susceptibility in mice after chronic treatment with apoptotic cells. However, induced IDO expression was restricted to CD169+ MZ MΦs in this model while DNA nanoparticles induced IDO expression in CD19+ DCs, indicating that apoptotic cells and DNA nanoparticles elicit similar but distinctive responses by MZ cells. Enhancing STING activation in MZ cells is an excellent mechanism to prevent or suppress autoimmunity.

### Example 16

### A Discrete Population of MZ DCs Produces IFNαβ after Sensing Nanoparticle Cargo DNA

### Materials and Methods

Transgenic mice expressing human diphtheria-toxin receptors (DTR) under the control of CD11c (CD11cDTR) or CD169 (CD169DTR) promoters were pre-treated with DT to deplete DCs or MZ MDs, respectively, before DNA nanoparticle treatment.

### Results

DC depletion reduced IFNαβ and IDO induction significantly, while depleting CD 169+ MZ MΦs had no significant effect on IDO induction and had little impact on IFNαβ induction by DNA nanoparticles (Table 3), indicating that DCs but not MZ MDs stimulated STING-dependent IFNαβ production and subsequent IDO induction in response to DNA nanoparticles.

### Example 17

### Materials and Methods

Innate immune cells were induced to express IFNβ1 by FACS and were sorted to obtain discrete splenocyte populations from B6 mice treated with DNA nanoparticles for 3 hours. IFNβ1 and β-actin gene transcription in sorted cells was evaluated by quantitative RTPCR analysis. Based on nanoparticle uptake data splenocytes were stained with CD11c and CD11b mAbs and sorted.

### Results

IFNβ1:β-actin transcript ratios were elevated substantially (10-20 fold) in unsorted splenocytes from mice treated with DNA nanoparticles, relative to IFNβ1:β-actin transcript ratios in splenocytes from untreated mice (FIG. 13, black bars). However, IFNβ1 transcripts were enriched consistently and substantially only in RNA samples from a small population of sorted cells that co-expressed high levels of CD11c and CD11b (CD11b⁺ DCs), which comprise only ˜2-5% of total CD11c⁺DCs (FIG. 13B, black bars). Induced IFNβ1 transcript levels were lower in all other sorted cell populations, relative to induced IFNβ1 transcript levels in unsorted splenocytes. Thus DNA nanoparticles stimulated rapid IFNβ1 gene transcription in rare splenic CD11b⁺ DCs, and did not elevate IFNβ1 transcription in sorted CD11cnegCD11b+(non-DCs), CD11c^{low}CD11b^{neg} (pDCs), CD11c^{high}CD11b^{low/neg} (myeloid DCs), or CD11c^{neg}CD¹¹b^{neg} cells (all other splenocytes). Increased IFNβ1 gene transcription was not detected in unsorted splenocytes, or in any cell population sorted from spleens of STING-KO mice after treatment (for 3 hrs) with DNA nanoparticles, indicating that STING activation by cargo DNA was responsible for increased IFNβ1 transcription (FIG. 13B, gray bars).

Collectively, the data herein show that DNA nanoparticles delivered cargo DNA rapidly to several discrete cell subsets located in splenic MZ, which therefore qualify as candidate cells that elicit robust regulatory responses via IFNαβ and IDO to activate Tregs. However only one cell type, a small population of CD11b⁺ DCs, responded to cargo DNA via STING-mediated cytoplasmic DNA sensing by up-regulating IFNβ1 gene transcription. Thus, STING activation in response to nanoparticle cargo DNA is cell-type specific. Since most cells are thought to express STING the basis of this highly selective response is unclear, but cell-type specificity may arise due to differential expression of cytoplasmic DNA sensors that trigger STING activation, or differential Trexl exonuclease activity that prevents STING activation by

degrading cytoplasmic DNA in some cells that ingest cargo DNA. This point notwithstanding our findings identify a small population of MZ DCs as pivotal cells able to promote robust regulatory responses to DNA nanoparticles when they sense cytoplasmic DNA and produce IFNαβ to activate IDO and Tregs.

### Example 18

### Nanoparticle Polymer Comparison

### Materials and Methods

DNA loaded nanoparticles composed of PEI or biodegradable β amino ester (C32) polymers complexed with bacterial plasmid DNA (pDNA) with (CpG+) or lacking (CpG^{free}) TLR9 ligands were evaluated to determine the effects of the different polymers on induced serum IFN type I and spleen IDO enzyme activity. B6 mice were injected with the nanoparticles as described above.

### Results

Table 4 shows that DNA nanoparticles composed of PEI or biodegradable β amino ester (C32) polymers induced serum IFN type I and spleen IDO enzyme activity with comparable efficiencies in mice.

TABLE 4
Polymer comparison

| | | |
|---|---|---|
| DNP treatment (i/v) | IFN type1^{a} | IDO activity^{b} |

| pDNA | Polymer | (serum) | (pmol/hr/mg) |
|---|---|---|---|
| — | — | <100 | <0.01 |
| CpG+ | PEI | 1980 | 1.9 |
| CpG+ | C32 | 2060 | 1.8 |
| CpG^{free} | C32 | 1460 | 1.3 |

^{a}Interferon bioassay (Units); nd, not detected
^{b}Kyn generated ex vivo from tissue homogenates

### Example 19

### DNA Nanoparticles Composed of (A) PEI or (B) Biodegradable β Amino Ester (C32) Polymers Induced Comparable Regulatory Phenotypes in Splenic Dendritic Cells and Regulatory T Cells (Tregs)

### Materials and Methods

DNA loaded nanoparticles made of PEI or biodegradable p amino ester (C32) polymers complexed with bacterial plasmid DNA (pDNA) containing (CpG+) or lacking (CpG^{free}) TLR9 ligands were injected into mice and regulatory responses were measured in several ways. Splenic dendritic cells (DCs, MACS-enriched CD11c⁺ cells) were removed after 24 hrs. and graded numbers of DCs were placed in culture with OVA-specific responder T cells from OT-1 T cell receptor transgenic (TCR-Tg) mice and cognate OVA peptide (SIINFEKL (SEQ ID NO:1), FIG. 15A) with or without IDO inhibitor (D-1MT). After 66 hrs culture ³H-thymidine was added and Thy incorporation was assessed (by counting TCA precipitable radioactivity) 6 hrs. later (FIG. 14).

### Results

DNA nanoparticles composed of PEI (FIG. 14A) or biodegradable β amino ester (C32) (FIG. 14B) polymers induced robust IDO-dependent regulatory phenotypes in splenic dendritic cells with comparable efficiencies in mice, as evidence by the absence of T cell proliferation, unless D-1MT was present in cultures.

### Example 20

### Materials and Methods

As depicted in the diagram shown in FIG. 15A marked (Thy1.1) and dye-labeled (CFSE) OVA-specific CD8 T cells from OT-1 TCR-Tg donor mice were injected into B6 recipient mice, which were immunized 24 hrs later (s.c to target inguinal lymph nodes) with OVA-expressing splenocytes from Act-mOVA-transgenic donor mice to elicit OT-1 responses in vivo. To assess the regulatory effects of DNPs, immunized mice containing OT-1 responder T cells were also treated with DNPs containing PEI or C32 polymers (and CpG^{free} pDNA) at the time of immunization (0 hrs), and again 48 hrs later. 72 hrs after OVA immunization numbers of cytolytic effector OT-1 T cell (Thy1.1, CD8+, GranzymeB+ cells) present in inguinal LNs were evaluated by flow cytometric analysis. Data were plotted as the mean numbers of cytolytic OT-1 T cells in inguinal LNs from triplicate mice in each group.

### Results

DNA nanoparticles (DNPs) composed of PEI or three different isoforms of biodegradable β amino ester (C32) polymers suppressed effector T cell responses to OVA at local immunization sites (inguinal lymph nodes) with comparable efficiencies in mice. FIG. 15A depicts the experimental design and FIG. 15B shows the number of OT-1 effector (GranzymeB⁺) T cells detected in inguinal LNs draining immunization sites. All DNP isoforms inhibited the generation of effector OT-1 T cells substantially, as evidenced by the much lower numbers of effector OT-1 T cell detected in inguinal LNs of DNP-treated mice relative to controls that received vehicle (Vh) instead. DNPs containing two C32 isoforms (C32-117, C32-118) were more effective in suppressing OT-1 responses in vivo than DNPs containing PEI, while DNPs containing a third C32 polymer (C32-122) were slightly less effective in suppressing OT-1 responses that DNPs containing PEI. These data demonstrate that DNPs containing biodegradable polymers were as effective as T cell supressors (or even more effective in some cases) than DNPs containing non-degradable PEI, which is not acceptable for clinical use.

We claim:

1. A method for inhibiting immune-mediated tissue destruction in a subject comprising administering to the subject an effective amount of a particulate formulation to inhibit or reduce immune-mediated tissue destruction in the subject compared to a control, wherein the particulate formulation comprises polymeric particles combined with polynucleotides and induces indoleamine 2,3 dioxygenase expression in the subject.

2. The method of claim 1, wherein the particulate formulation induces Tregs.

3. The method of claim 1, wherein the polynucleotides comprise bacterial plasmid DNA.

4. The method of claim 1, wherein particulate formulation comprises a final nitrogen residues:nucleic acid phosphate (N:P) ratio of 10 to 18.

5. The method of claim 1, wherein the polymeric particles comprise polymer polyethylenimine.

6. The method of claim 5, wherein the PEI is linear, circular, branched, super coiled, single-stranded, or double-stranded.

7. The method of claim 1, wherein the polymeric particles comprise the bio-degradable polymer poly beta amino ester and derivatives thereof.

8. The method of claim 1, wherein the particulates comprise nanoparticles, microparticles, or a combination thereof.

9. A method for treating an autoimmune disease in a subject comprising administering to the subject an effective amount of a particulate formulation to inhibit or reduce one or more symptoms of an autoimmune disease in the subject compared to a control, wherein the particulate formulation particles in combination with polynucleotides.

10. The method of claim 9 wherein the autoimmune disease is selected from the group consisting of rheumatoid arthritis, systemic lupus erythematosus, alopecia greata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

11. A method for enhancing the effect of a tolerizing vaccine in a subject comprising administering to the subject a tolerizing vaccine in combination with an effective amount of a particulate formulation to enhance the effect of a tolerizing vaccine compared to a control, wherein the particulate formulation comprises particles in combination with polynucleotides.

12. A method for treating asthma in a subject comprising administering to the subject an effective amount of a particulate formulation to inhibit or reduce one or more symptoms of asthma in a subject compared to a control, wherein the particulate formulation comprises particles in combination with polynucleotides.

13. The method of claim 12 wherein the symptom of asthma is lung inflammation.

14. A method for enhancing the effect of mucosal tolerance in a subject comprising administering to the subject an antigen in combination with an effective amount of a particulate formulation to enhance the effect of mucosal tolerance in a subject compared to a control, wherein the particulate formulation comprises particles in combination with polynucleotides, wherein the antigen is administered to a mucosa.

15. The method of claim 14 wherein the mucosa is selected from the group consisting of oral, nasal, and gastrointestinal.

16. The method according to any one of claims 9-15, wherein expression of IDO enzyme activity is stimulated in the subject.

17. The method of claim 16 wherein IDO enzyme activity is stimulated at sites of inflammation.

18. The method of claim 16 wherein the IDO is expressed in cells selected from the group consisting of fibroblasts, dendritic cells, macrophages, and epithelial cells.

19. The method of claim 18 wherein the dendritic cells display attributes of plasmacytoid DCs (pDCs) or B cells.

20. The method of claim 18 wherein the dendritic cells are CD19+, Pax5+, CD11c+, or combination thereof.

21. The method according to any one of claims 1-19, wherein systemic release of one or more proinflammatory molecules is reduced relative to a control.

22. The method according to any one of claims 1-21, wherein systemic IFNγ is reduced relative to a control.

23. The method according to any one of claims 1-22, wherein systemic activation of natural killer cells in the subject is reduced relative to a control.

24. The method according to any one of claims 1-23, wherein differentiation, activation, or proliferation of effector T cells is reduced relative to a control.

25. The method according to any one of claims 1-22, wherein Tregs are induced to acquire or enhance a suppressor function relative to a control.

26. The method of claim 25 wherein the suppressor function is selected from the group consisting of exhibit increased proliferation, and enhanced production of IL-10, IL-2 and TGF-β.

27. A method for inducing indoleamine 2,3 dioxygenase IDO-dependent regulatory phenotypes in cells comprising contacting the cells with an effective amount of a particulate formulation comprising particles in combination with polynucleotides to induce IDO-dependent regulatory phenotypes in the cells.

28. The method of claim 27 wherein the contacting occurs in vivo or ex vivo.

29. The method of claim 27 wherein the contacting occurs ex vivo and the cells are administered to a subject to induce an immune suppressive response in a subject.

30. The method according to any one of claims 1-29 wherein unmethylated CpG motifs are masked or absent on the polynucleotides.

31. The method according to any one of claims 1-29 wherein CpG motifs are masked or absent on the polynucleotides.

32. The method according to any one of claims 1-29 wherein TLR9 ligands are masked or absent on the polynucleotides.

33. The method according to any one of claims 1-31 wherein immunostimulatory elements are masked or absent on the polynucleotides.

34. The method according to any one of claims 1-29 wherein the polynucleotides comprise polyA:T.

35. A composition comprising an effective amount of a particulate formulation to enhance or promote immune tolerance in a subject, wherein the particulate formulation comprises particles in combination with non-coding polynucleotides.

36. The composition of claim 35 wherein the particulate formulation can induce expression of IDO enzyme activity in IDO competent cells.

37. The composition according to any one of claims 35-36 wherein systemic release of one or more proinflammatory molecules is reduced relative to a control when the composition is administered to a subject.

38. The composition according to any one of claims 35-37 wherein systemic IFNγ is reduced relative to a control when the composition is administered to a subject.

39. The composition according to any one of claims 35-38 wherein systemic activation of natural killer cells reduced relative to a control when the composition is administered to a subject.

40. The composition according to any one of claims 35-39, wherein differentiation, activation, or proliferation of effector T cells is reduced relative to a control when the composition is administered to a subject.

41. The composition according to any one of claims 35-40, wherein Tregs are induced to acquire or enhance a suppressor function relative to a control when the composition is administered to a subject.

42. The composition according to any one of claims 35-41 wherein the suppressor function is selected from the group consisting of exhibit increased proliferation, and enhanced production of IL-10, IL-2 and TGF-β when the composition is administered to a subject.

43. The composition according to any one of claims 35-42 wherein unmethylated CpG motifs are masked or absent on the polynucleotides.

44. The composition according to any one of claims 35-43 wherein CpG motifs are masked or absent on the tolerogenic polynucleotides.

45. The composition according to any one of claims 35-44 wherein TLR9 ligands are masked or absent on the polynucleotides.

46. The composition according to any of claims 35-45 wherein immunostimulatory elements are masked or absent on the polynucleotides.

47. The composition according to any one of claims 35-46 wherein the polynucleotides comprise polyA:T polynucleotides.


SEQUENCE LISTING (2 sequences)

SEQ ID NO: 1 - protein, 8 aa, Artificial Sequence
  Synthetic OVA peptide
SIINFEKL

SEQ ID NO: 2 - protein, 15 aa, Artificial Sequence
  Synthetic H-Y peptide
REEALHQFRSGRKPI